(19) United States
(12) Patent Application Publication
(10) Pub. No.: US 2023/0049822 A1
(43) Pub. Date: Feb. 16, 2023

(54) COMPOSITIONS COMPRISING NICLOSAMIDE FOR USE IN TREATING CONDITIONS ASSOCIATED WITH AN ABNORMAL INFLAMMATORY RESPONSE

(71) Applicant: First Wave Bio, Inc., Ann Arbor, MI (US)

(72) Inventor: Gary D. Glick, Ann Arbor, MI (US)

(21) Appl. No.: 17/791,639

(22) PCT Filed: Jan. 8, 2021
(86) PCT No.: PCT/US2021/012675

§ 371 Date: Jul. 8, 2022

Related U.S. Application Data
(60) Provisional application No. 62/959,423, filed on Jan. 10, 2020.

(51) Int. Cl. A61K 9/00; A61K 31/167; A61K 9/10; A61P 1/04

(52) U.S. Cl. CPC A61K 9/0031; A61K 31/167; A61K 9/10; A61P 1/04

(57) ABSTRACT

This disclosure features compositions comprising niclosamide for use in treating one or more conditions (or one or more symptoms thereof) characterized by an abnormal inflammatory response in one or more particular subject (e.g., patient) populations in need thereof. Such conditions include, e.g., an autoimmune disorder, e.g., colitis, e.g., autoimmune colitis, e.g, an inflammatory bowel disease, e.g., Crohn's disease, ulcerative colitis. In some embodiments, the uses include rectally (e.g., via enema) administering niclosamide.

### CROSS REFERENCE TO RELATED APPLICATIONS

This application claims the benefit of U.S. Provisional Application Ser. No. 62/959,423, filed on Jan. 10, 2020, the disclosure of which is incorporated herein by reference in its entirety.

### TECHNICAL FIELD

This disclosure features methods for treating one or more conditions (or one or more symptoms thereof) characterized by an abnormal inflammatory response in one or more particular subject (e.g., patient) populations in need thereof. Such conditions include, e.g., an autoimmune disorder, e.g., colitis, e.g., autoimmune colitis, e.g, an inflammatory bowel disease, e.g., Crohn's disease, ulcerative colitis). The methods include administering to the subject an effective amount of niclosamide or a pharmaceutically acceptable salt and/or cocrystal thereof as well as compositions containing the same. In some embodiments, the methods include rectally (e.g., via enema) administering niclosamide.

### BACKGROUND

Ulcerative colitis (UC) and Crohn's disease (CD) are the predominant chronic, inflammatory bowel diseases (IBD) in humans. These disorders are autoimmune in nature and occur in the absence of infection. IBD effects up to 2,000,000 Americans (increasing ˜15% annually) and it is associated with unacceptably high rates of morbidity and mortality. IBD is also a significant burden on the U.S. health care system as the most effective treatments are biological drugs that are quite costly.

IBD occurs as the result of inappropriate immune responses in genetically susceptible individuals mediated by complex interactions between environmental stimuli, microbial factors, and the intestinal immune system. The hallmark of IBD is represented by excessive immune responses that mediate gastrointestinal tissue damage, either directly or through the release of soluble, pro-inflammatory mediators.

T cells are a type of immune cell that infiltrate the intestinal mucosa and are key drivers of gastrointestinal tissue damage in IBD. These cells persist and accumulate in the intestinal mucosa because normal physiologic mechanisms designed to censor or eliminate activated T cells are inoperative in the context of IBD. While the exact basis for T cell accumulation in IBD is not fully elucidated, chronic activation by microbial stimuli along with the cytokine milieu at the sites of inflammation within gastrointestinal tissue are thought to be important. Regardless of how these cells persist, enhancing T cell death in the intestinal mucosa is linked with resolution of IBD and drugs that are most effective in managing IBD function (in part), by killing pathogenic T cells resident in the gut.

Although different forms of IBD show pathophysiological and clinical differences, the therapeutic approach to managing IBD shares many common elements. Medical management of IBD is largely empirical, employing anti-inflammatory or immunosuppressive drugs. Salicylazosulfapyridine and 5-aminosalicylic acid are used to treat mild IBD and as maintenance therapy if disease remission can be achieved. Corticosteroids are used in patients with moderate to severe disease. However, clinical remission can only be obtained in ˜60% of patients, and just about half of these stay in remission after treatment is discontinued. This last point is significant because long-term use of corticosteroids carries a significant risk of serious side effects.

Immunosuppressive drugs can also be used to treat moderate to severe cases of IBD, often as a replacement for steroid therapy. However, immunosuppressive drugs (e.g., azathioprine) usually cannot ensure control of symptoms, and treatment is accompanied by numerous contraindications and severe side effects.

Drugs that often show the best efficacy in treating IBD are systemically administered (via injection or infusion) monoclonal antibodies that block TNF-alpha, a pro-inflammatory cytokine overproduced during all forms of IBD (e.g., UC, CD, graft-versus-host disease, celiac disease, iatrogenic colitis such as that induced by checkpoint inhibitors, etc.). Reducing levels of TNF-alpha in the context of IBD has two consequences. First, as an inflammatory cytokine, TNF-alpha mediates tissue damage. Second, high levels of TNF-alpha help disease causing T cells to survive and blocking TNF-alpha activity eventually leads to T cell death. Indeed, the induction of cell death by anti-TNF-alpha drugs like infliximab can predict clinical improvement in patients.

Although effective, use of anti-TNF-alpha drugs is associated with severe, systemic side effects including, re-activation of latent pathogens, hypersensitivity phenomena, cancer, and the formation of autoantibodies. Some patients are inherently resistant to anti-TNF-alpha drugs and over time, almost half of all patients that do show a response, develop resistance.

From the foregoing it is clear that there is need for new drugs to treat IBD that are more effective, less toxic, less expensive, and more convenient to administer versus standard of care.

Niclosamide (5-chloro-N-(2-chloro-4-nitrophenyl)-2-hydrobenzamide) is a halogenated salicylanilide that belongs to a group of medicines known as anthelmintics. Anthelmintics are medicines used in the treatment of worm infections. Niclosamide, which has low systemic bioavailability and an excellent safety profile, is used to treat broad or fish tapeworm, dwarf tapeworm, and beef tapeworm infections. It is believed that Niclosamide inhibits oxidative phosphorylation and stimulates adenosine triphosphatase activity in the mitochondria of cestodes (e.g., tapeworm), killing the scolex and proximal segments of the tapeworm both in vitro and in vivo (see, Li, Y., et al., Cancer Lett. 2014 349, 8-14).

Recent studies have also identified other potential uses of niclosamide; e.g., as a potential anticancer agent (Id.); and as an agent for treating, preventing and/or alleviating the symptoms of type II diabetes and diabetes-related disorders or complications (see, e.g., WO 2012/068274). U.S. Pat. No. 8,148,328 discloses that niclosamide enhances the oral bioavailability of certain peptides.

### SUMMARY

This disclosure features methods for treating one or more conditions (or one or more symptoms thereof) characterized by an abnormal inflammatory response in one or more particular subject (e.g., patient) populations in need thereof. Such conditions include, e.g., an autoimmune disorder, e.g., colitis, e.g., autoimmune colitis, e.g, an inflammatory bowel disease, e.g., Crohn's disease, ulcerative colitis). The methods include administering to the subject an effective amount of niclosamide or a pharmaceutically acceptable salt and/or cocrystal thereof as well as compositions containing the same. In some embodiments, the methods include rectally (e.g., via enema) administering niclosamide.

This disclosure is based, in part, on the finding that niclosamide kills pathogenic T cells isolated from IBD patients and is effective in murine models of IBD. While not wishing to be bound by theory, it is believed that niclosamide or a pharmaceutically acceptable salt and/or hydrate and/or cocrystal thereof uncouple mitochondrial respiration from oxidative phosphorylation in one or more T cells, thereby disrupting the mitochondrial energy cycle in the one or more T cells and inducing cell death of the one or more T cells (e.g., activated T cells). It has been shown that niclosamide selectively targets and kills T cells associated with pathologies characterized by an abnormal inflammatory response (e.g., pathogenic T cells in the intestinal mucosa). See, e.g., WO 2017/040864, which is incorporated herein by reference in its entirety.

In embodiments, the methods described herein can be carried out using one or more of the following compositions or formulations.

In one example, a solid pharmaceutical composition, which comprises: an inner phase which is a wet granulated solid preparation comprising niclosamide, or a pharmaceutically acceptable salt thereof, one or more disintegrants; one or more diluents; and one or more binders; and an external phase comprising one or more glidants and/or one or more lubricants.

In another example, a solid pharmaceutical composition, which comprises: an inner phase which is a wet granulated solid preparation comprising niclosamide, or a pharmaceutically acceptable salt thereof; crospovidone; lactose monohydrate; and povidone; and an external phase comprising magnesium stearate and talc.

In a further example, an enema preparation comprising a separately contained first component and a separately contained second component, wherein: (i) the first component comprises a solid pharmaceutical composition, which comprises: an inner phase which is a wet granulated solid preparation comprising niclosamide, or a pharmaceutically acceptable salt thereof; one or more disintegrants; one or more diluents; and one or more binders; an external phase comprising one or more glidants and/or one or more lubricants; and (ii) the second component comprises one or more liquids and optionally one or more other pharmaceutically acceptable excipients together forming a liquid carrier.

In another aspect, provided herein is an enema formulation comprising water; methyl cellulose; povidone; methylparaben; propylparaben; sodium dihydrogen phosphate dehydrate; disodium phosphate dodecahydrate; crospovidone; lactose monohydrate; magnesium stearate; talc; and niclosamide, or a pharmaceutically acceptable salt thereof.

Accordingly, in one aspect, this disclosure features a method for treating colitis in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier. In certain embodiments, the colitis is ulcerative colitis, such as ulcerative proctitis or ulcerative proctosigmoiditis.

In another aspect, provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier.

In another aspect, provided herein is a method for treating a condition (or one or more symptoms thereof) characterized by an abnormal inflammatory response in a subject in need thereof are provided (e.g., an autoimmune disorder, e.g., an inflammatory bowel disease) in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier.

In some embodiments of one or more of the methods herein, the subject has a modified Mayo score (MMS) equal to from 4 points or greater than 4 points to 8 points or less than 8 points prior to said administering.

In some embodiments, the subject has at least one of (e.g., 1, 2, 3, or 4 of):
(I) a modified Mayo score (MMS) following said administering that is at least 2 points lower than a modified Mayo score (MMS) in the subject prior to said administering;
(II) a modified Mayo score (MMS) following said administering that is at least 25% lower than a modified Mayo score (MMS) in the subject prior to said administering;
(III) a Rectal Bleeding Score (RBS) following said administering that is at least 1 point lower than a Rectal Bleeding Score (RBS) in the subject prior to said administering; or
(IV) a Rectal Bleeding Score (RBS) following said administering that is 0 or 1.

In some embodiments of one or more of the methods herein, the subject does not have a modified endoscopic subscore of 0 or 1 prior to said administering and the subject has a modified endoscopic subscore of 0 or 1 following said administering.

In some embodiments of one or more of the methods herein, following said administering the subject shows an endoscopic mucosal healing of at least 20%, such as at least 30%, such as at least 40%, relative to prior to said administering.

In some embodiments of one or more of the methods herein, following said administering the subject shows a decrease in the level of an inflammatory marker relative to the level of the inflammatory marker prior to said administering.

In another aspect, provided herein is a method for:

A) inducing cell death of one or more T cells as disclosed herein (e.g., in the digestive and/or gastrointestinal tract (GI), skin, eyes, or joints), of a subject in need thereof; or

B) a treating a subject having a condition associated with unregulated (abnormal, elevated) recruitment and/or retention of one or more T cells as disclosed herein (e.g., at the digestive and/or gastrointestinal tract (GI), skin, eyes, or joints) of a subject;

the method comprising contacting the one or more T cells with an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier.

In some embodiments, the subject has a modified Mayo score (MMS) equal to from 4 points or greater than 4 points to 8 points or less than 8 points prior to said contacting.

In some embodiments, the subject has at least one of:
a) a modified Mayo score (MMS) following said contacting that is at least 2 points lower than a modified Mayo score (MMS) in the subject prior to said contacting;
b) a modified Mayo score (MMS) following said contacting that is at least 25% lower than a modified Mayo score (MMS) in the subject prior to said contacting;
c) a Rectal Bleeding Score (RBS) following said contacting that is at least 1 point lower than a Rectal Bleeding Score (RBS) in the subject prior to said contacting; or
d) a Rectal Bleeding Score (RBS) following said contacting that is 0 or 1.

In some embodiments, the subject does not have a modified endoscopic subscore of 0 or 1 prior to said contacting and the subject has a modified endoscopic subscore of 0 or 1 following said contacting.

In some embodiments, following said contacting the subject shows an endoscopic mucosal healing of at least 20%, such as at least 30%, such as at least 40%, relative to prior to said contacting.

In some embodiments, following said contacting the subject shows a decrease in the level of an inflammatory marker relative to the level of the inflammatory marker prior to said contacting.

In some embodiments, the methods described herein can provide targeted delivery of niclosamide to certain regions of the GI tract (e.g., the colon, e.g., the ascending colon and/or the transverse colon and/or the distal colon (e.g., for treatment of an inflammatory bowel disease such as ulcerative colitis); e.g., the small bowel, e.g., the ileum (e.g., for treatment of an inflammatory bowel disease such as Crohn's disease)). In some embodiments, administration (e.g., rectal administration) of niclosamide described herein to a subject produces a local concentration of niclosamide in the GI tract (e.g., colon, e.g., supra; e.g., the small bowel, e.g., the ileum) of the subject that is higher than the concentration of niclosamide in the plasma compartment of the subject, thereby, e.g., more efficiently providing niclosamide to diseased tissue in the GI tract (e.g., supra) and reducing risks associated with high systemic niclosamide exposure (e.g., toxicity). Moreover, the foregoing can potentially be achieved using a lower dosage of niclosamide.

The methods and compositions described herein not only provide treatment options that are highly efficient and effective at killing T cells, but also ones that address the toxicity, cost, and convenience issues associated with some standard methods of treatment.

In embodiments, the methods described herein can be carried out using niclosamide, a small molecule that has an established and good safety profile and is an FDA approved anthelmintic drug.

The methods and compositions described herein are also expected to be functional in diverse patient populations and/or less sensitive to blocks in cell death mechanisms. Further, the ability to utilize traditional small molecules, such as niclosamide, can help reduce cost and facilitate patient administration.

In some embodiments, the methods and compositions described herein are suitable for use in combination therapy with various other therapeutic regimens (e.g., chemotherapy and/or radiation). In certain embodiments, the methods described herein can be used to treat side effects produced by such therapeutic regimens, e.g., inflammatory bowel diseases induced by chemotherapeutic immunomodulators, e.g., checkpoint inhibitors, which in some cases can be prohibitively severe. Additionally, the chemical entities, methods, and compositions described herein are also expected to be useful in certain treatment-resistant patient populations, e.g., one that is nonresponsive or resistant to treatment an anti-TNFalpha therapy (e.g., Humira, Enbrel, Remicade).

Embodiments can include one or more of the following features.

The condition can be associated with unregulated (such as abnormal or elevated) recruitment and/or retention of one or more T cells at the gastrointestinal tract (GI) of the subject.

The condition can be associated with unregulated (such as abnormal or elevated) activation of one or more T cells in the gastrointestinal tract (GI) of the subject.

The condition can be colitis. For example, the condition can be an autoimmune colitis. The condition can be an inflammatory bowel disease (e.g., ulcerative colitis (such as ulcerative proctitis or ulcerative proctosigmoiditis) or Crohn's disease). The condition can be iatrogenic autoimmune colitis.

The condition can be colitis (e.g., iatrogenic autoimmune colitis) induced by one or more chemotherapeutic agents.

At least one of the one or more chemotherapeutic agents can be a chemotherapeutic immunomodulator such as an immune checkpoint inhibitor. The immune checkpoint inhibitor can be an inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine—TIM3, lymphocyte activation gene 3 protein (LAG3), MHC class II-LAG3, 4-1BB-4-1BB ligand, OX40-OX40 ligand, GITR, GITR ligand—GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine—TIM3, SIRPA-CD47, VEGF, Neuropilin, CD160, CD30, and CD155. The immune checkpoint inhibitor can be selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A, and MGA271. The immune checkpoint inhibitor can be an inhibitor that targets CTLA-4. The immune checkpoint inhibitor can be an antibody. The antibody can be is ipilimumab or tremelimumab. The immune checkpoint inhibitor can be an inhibitor that targets PD1 or PD-L1. The immune checkpoint inhibitor can be selected from nivolumab, lambrolizumab, and BMS-936559.

The condition can be selected from the group consisting of celiac disease, irritable bowel syndrome, mucositis, uveitis, radiation enteritis, rheumatoid arthritis, lupus, scleroderma, psoriasis, cutaneous T-cell lymphoma, acute graft vs. host disease and chronic graft vs. host disease.

The methods can further include administering one or more additional therapeutic agents. For example, therapeutic agents useful for treating or preventing inflammatory bowel disease (IBD) (e.g., Crohn's disease, ulcerative colitis), e.g., sphingosine 1-phosphate (S1P) receptor modulators (e.g., etrasimod or ozanimod); steroidal anti-inflammatory agents (e.g, beclomethasone 17 or budesonide); non-steroidal anti-inflammatory agents (e.g., 5-ASA); receptor-interacting protein kinase 1 (RIPK1) inhibitors (e.g., GSK2982772); EP4 modulators (e.g., KAG-308); toll-like receptor (e.g., TLR4, TLR9) modulators (e.g., JKB-122, cobitolimod); Janus kinase (JAK) inhibitors (e.g., TD-1473, tofacitinib, upadacitinib, filgotinib, PF-06651600, and PF-06700841); lanthionine synthetase C-like 2 (LANCL2) modulators (e.g., BT-11); phosphatidylcholine (e.g., LT-02); integrin (e.g., α4 Integrin) modulators (e.g, AJM-300 (carotegrast)); Smad7 modulators (e.g., mongersen); phosphodiesterase 4 (PDE4) modulators (e.g., apremilast); tumor progression locus 2 (TPL2) inhibitors (e.g., GS-4875); tyrosine kinase 2 (TYK2) inhibitors (e.g., BMS-986165, PF-06700841, and PF-06826647); and/or TEC kinase inhibitors (e.g., PF-06651600).

As another example, the one or more therapeutic agents can be: budesonide; epidermal growth factor; corticosteroids; cyclosporine; sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1 monoclonal antibodies; anti-IL-6 monoclonal antibodies (e.g., anti-IL-6 receptor antibodies and anti-IL-6 antibodies); growth factors; elastase inhibitors; pyridinyl-imidazole compounds; TNF antagonists as described herein; IL-4, IL-10, IL-13 and/or TGF.beta. cytokines or agonists thereof (e.g., agonist antibodies); IL-11; glucuronide- or dextran-conjugated prodrugs of prednisolone, dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of platelet activating factor (PAF); ciprofloxacin; and/or lignocaine.

As a further example, the one or more additional therapeutic agents can be therapeutic agents and/or regimens for treating autoimmune colitis. Non-limiting examples corticosteroids (e.g., budesonide, prednisone, prednisolone, Beclometasone dipropionate), diphenoxylate/atropine, infliximab, loperamide, mesalamine, TIP60 inhibitors (see, e.g., U.S. Patent Application Publication No. 2012/0202848), and vedolizumab.

As a further example, the one or more additional therapeutic agents can be therapeutic agents and/or regimens for treating iatrogenic autoimmune colitis. Non-limiting examples include corticosteroids (e.g., budesonide, prednisone, prednisolone, Beclometasone dipropionate), diphenoxylate/atropine, infliximab, loperamide, TIP60 inhibitors (see, e.g., U.S. Patent Application Publication No. 2012/0202848), and vedolizumab.

As a further example, the one or more additional therapeutic agents can be therapeutic agents and/or regimens for treating colitis induced by one or more chemotherapeutics agents. Non-limiting examples include corticosteroids (e.g., budesonide, prednisone, prednisolone, beclometasone dipropionate), diphenoxylate/atropine, infliximab, loperamide, mesalamine, TIP60 inhibitors (see, e.g., U.S. Patent Application Publication No. 2012/0202848), and vedolizumab.

As a further example, the one or more additional therapeutic agents can be therapeutic agents and/or regimens for treating colitis induced by treatment with adoptive cell therapy. Non-limiting examples include corticosteroids (e.g., budesonide, prednisone, prednisolone, beclometasone dipropionate), diphenoxylate/atropine, infliximab, loperamide, TIP60 inhibitors (see, e.g., U.S. Patent Application Publication No. 2012/0202848), and vedolizumab.

As a further example, the one or more additional therapeutic agents can be therapeutic agents and/or regimens for treating colitis associated with one or more alloimmune diseases. Non-limiting examples include corticosteroids (e.g., budesonide, prednisone, prednisolone, beclometasone dipropionate), sulfasalazine, and eicopentaenoic acid.

As a further example, the one or more additional therapeutic agents can be therapeutic agents and/or regimens for treating radiation enteritis. Non-limiting examples include teduglutide, amifostine, angiotensin-converting enzyme (ACE) inhibitors (e.g., benazepril, captopril, enalapril, fosinopril, lisinopril, moexipril, perindopril, quinapril, ramipril, and trandolapril), probiotics, selenium supplementation, statins (e.g., atorvastatin, fluvastatin, lovastatin, pravastatin, rosuvastatin, simvastatin, and pitavastatin), sucralfate, and vitamin E.

As a further example, the one or more additional therapeutic agents can be therapeutic agents and/or regimens for treating collagenous colitis. Non-limiting examples include 6-mercaptopurine, azathioprine, bismuth subsalicate, Boswellia serrata extract, cholestyramine, colestipol, corticosteroids (e.g., budesonide, prednisone, prednisolone, beclometasone dipropionate), loperamide, mesalamine, methotrexate, probiotics, and sulfasalazine.

As a further example, the one or more additional therapeutic agents can be therapeutic agents and/or regimens for treating lyphocytic colitis. Non-limiting examples include 6-mercaptopurine, azathioprine, bismuth subsalicylate, cholestyramine, colestipol, corticosteroids (e.g., budesonide, prednisone, prednisolone, beclometasone dipropionate), loperamide, mesalamine, methotrexate, and sulfasalazine.

As a further example, the one or more additional therapeutic agents can be therapeutic agents and/or regimens for treating microscopic colitis. Non-limiting examples include 6-mercaptopurine, azathioprine, bismuth subsalicylate, Boswellia serrata extract, cholestyramine, colestipol, corticosteroids (e.g., budesonide, prednisone, prednisolone, beclometasone dipropionate), fecal microbial transplantation, loperamide, mesalamine, methotrexate, probiotics, and sulfasalazine.

As a further example, the one or more additional therapeutic agents can be therapeutic agents and/or regimens for treating UC. Non-limiting examples include AbGn-168H, ABT-494, ABX464, apremilast, PF-00547659, PF-06687234, 6-mercaptopurine, adalimumab, azathioprine, bertilimumab, brazikumab (MEDI2070), cobitolimod, certolizumab pegol (Cimzia®), CP-690,550, corticosteroids (e.g., multimax budesonide, Methylprednisolone), cyclosporine, E6007, etrasimod, etrolizumab, fecal microbial transplantation, filgotinib, guselkumab, golimumab, IL-2, IMU-838, infliximab, matrix metalloproteinase 9 (MMP9) inhibitors (e.g., GS-5745), mesalamine, mesalamine, mirikizumab (LY3074828), RPC1063, risankizumab (BI 6555066), SHP647, sulfasalazine, TD-1473, TJ301, tildrakizumab (MK 3222), tofacitinib, tofacitinib, ustekinumab, UTTR1147A, and vedolizumab.

As a further example, the one or more additional therapeutic agents can be therapeutic agents and/or regimens for treating Crohn's Disease (CD). Non-limiting examples include adalimumab, autologous CD34-selected peripheral blood stem cells transplant, 6-mercaptopurine, azathioprine, certolizumab pegol (Cimzia®), corticosteroids (e.g., prednisone), etrolizumab, E6011, fecal microbial transplantation, filgotinib, guselkumab, infliximab, IL-2, JAK inhibitors, matrix metalloproteinase 9 (MMP 9) inhibitors (e.g., GS-5745), MEDI2070, mesalamine, methotrexate, natalizumab, ozanimod, RHB-104, rifaximin, risankizumab, SHP647, sulfasalazine, thalidomide, upadacitinib, V565, and vedolizumab.

As a further example, the one or more additional therapeutic agents can be therapeutic agents and/or regimens for treating IBDs. Non-limiting examples include 6-mercaptopurine, AbGn-168H, ABX464, ABT-494, adalimumab, AJM300, alicaforsen, AMG139, anrukinzumab, apremilast, ATR-107 (PF0530900), autologous CD34-selected peripheral blood stem cells transplant, azathioprine, bertilimumab, BI 655066, BMS-936557, certolizumab pegol (Cimzia®), cobitolimod, corticosteroids (e.g., prednisone, Methylprednisolone, prednisone), CP-690,550, CT-P13, cyclosporine, DIMS0150, E6007, E6011, etrasimod, etrolizumab, fecal microbial transplantation, filgotinib, fingolimod, firategrast (SB-683699) (formerly T-0047), GED0301, GLPG0634, GLPG0974, guselkumab, golimumab, GSK1399686, HMPL-004 (Andrographis paniculata extract), IMU-838, infliximab, Interleukin 2 (TL-2), Janus kinase (JAK) inhibitors, laquinimod, masitinib (AB1010), matrix metalloproteinase 9 (MMP 9) inhibitors (e.g., GS-5745), MEDI2070, mesalamine, methotrexate, mirikizumab (LY3074828), natalizumab, NNC 0142-0000-0002, NNC0114-0006, ozanimod, peficitinib (JNJ-54781532), PF-00547659, PF-04236921, PF-06687234, QAX576, RHB-104, rifaximin, risankizumab, RPC1063, SB012, SHP647, sulfasalazine, TD-1473, thalidomide, tildrakizumab (MK 3222), TJ301, TNF-Kinoid®, tofacitinib, tralokinumab, TRK-170, upadacitinib, ustekinumab, UTTR1147A, V565, vatelizumab, VB-201, vedolizumab, and vidofludimus.

### Definitions

To facilitate understanding of the disclosure set forth herein, a number of terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “Mayo score” (MMS) refers to a composite score assigned to a subject. The score is the sum of the following 4 subscores:
(i) Stool Frequency (SF): The SF subscore is a patient-reported measure. This item reports the number of stools in a 24-hour period, relative to the normal number of stools for that patient in the same period, on a 4-point scale. A stool is defined as a trip to the toilet when the patient has either a bowel movement, or passes blood alone, blood and mucus, or mucus only. The total number of stools passed in a 24-hour period is recorded by the patient. The reference “normal” SF for that patient is typically recorded at the outset of a study or period of observation. Normal SF for that patient is on the reported SF when the patient was in remission or, if the patient has never achieved remission, the reported SF before initial onset of signs and symptoms of ulcerative colitis;
(ii) Rectal Bleeding (RB): The RB subscore is a patient-reported measure. This item reports the most severe amount of blood passed per rectum for a given day, on a 4-point scale;
(iii) Endoscopic Subscore (ES): The ES is a physician-reported measure that reports the worst appearance of the mucosa on flexible sigmoidoscopy or colonoscopy, on a 4-point scale. Consistent with current clinical practice, friability is excluded from the definition of an ES of 1; and
(iv) Physician's Global Assessment (PGA): The PGA is a physician-reported measure that summarizes the assessment of the patient's disease activity on a 4-point scale.

Each subscore is scored on a 4-point scale, ranging from 0 to 3 as shown below, to give a maximum Mayo score of 12.

| Stool Frequency Subscore | Score |
|---|---|
| Normal number of stools for subject | 0 |
| 1 to 2 stools more than normal | 1 |
| 3 to 4 stools more than normal | 2 |
| 5 or more stools than normal | 3 |

| Rectal Bleeding Subscore | Score |
|---|---|
| No blood seen | 0 |
| Streaks of blood with stool | 1 |
| less than half of the time | |
| Obvious blood (more than just streaks) or | 2 |
| streaks of blood with stool most of the | |
| time | |
| Blood alone passed | 3 |

| Endoscopic Subscore | Score |
|---|---|
| Normal or inactive disease | 0 |
| Mild disease (erythema, | 1 |
| decreased vascular pattern) | |
| Moderate disease (marked erythema, | 2 |
| absent vascular pattern, friability, erosions) | |
| Severe disease (spontaneous bleeding, | 3 |
| ulceration) | |

| Physician’s Global Assessment | Score |
|---|---|
| Normal | 0 |
| Mild disease | 1 |
| Moderate disease | 2 |
| Severe disease | 3 |

The modified Mayo score (MMS) is a modification made to the original Mayo Index reference (Schroeder et aL, New Eng J Med, 317(26): 1625-1629, 1987 which is incorporated herein by reference in its entirety). The MMS includes 3 of the 4 types of subscores of the Mayo Score (see Inflamm Bowel Dis. 2008; 14(12):1660-6; and BMC Gastroenterology (2018) 18:173, each of which is incorporated herein by reference in its entirety). It does not include the Physician's Global Assessment. The MMS evaluates three subscores, each on a scale of 0 to 3 with a maximum total score of 9. The following table summarizes the respective MMS subscales for scoring.

Modified Mayo Score (MMS)

| | | Rectal Bleeding | Endoscopy Score |
|---|---|---|---|
| Index | Stool Frequency (SF) | (RB) | (ES) |
| MMS | 0-Normal number | 0-No blood seen | 0-normal or |
| | of stools per day for | 1 = streaks of | inactive disease |
| | this patient | blood with stool | 1 = mild disease |
| | 1 = 1 to 2 more stools | less than half the | (erythema |
| | per day for this | time | decreased vascular |
| | patient | 2 = obvious blood | pattern) |
| | 2 = 3 to 4 more stools | with stool most of | 2 = moderate |
| | than normal | the time | disease (marked |
| | 3 = 5 or more stools | 3 = blood alone | erythema, absent |
| | than normal | passed | vascular pattern, |
| | | | friability erosions) |
| | | | 3 = severe disease |
| | | | (spontaneous |
| | | | bleeding, |
| | | | ulceration) |

As used herein, the term “rectal bleeding score” (RBS) refers to the rectal bleeding score of the MMS as defined supra.

As used herein, the term “modified endoscopic subscore” refers to endoscopy score of the MMS as defined supra.

“Endoscopic mucosal healing”, as used herein, refers to endoscopic healing and histologic healing (see “mucosal healing” in Sands B E, Sandborn W J, Panaccione R, et al. Ustekinumab as induction and maintenance therapy for ulcerative colitis. N Engl J Med 2019; 381:1201-14 and supplementary materials therefor which are incorporated herein by reference in their entireties). Endoscopic healing is based on the improvement in the endoscopic appearance of the mucosa (ibid.). It can be indicated by the endoscopy subscore of the Mayo score or modified Mayo score (MMS) (e.g., Mayo score=0 or 1).

As used herein, the term “histologic improvement” refers to histologic healing as defined in Sands B E, Sandborn W J, Panaccione R, et al. Ustekinumab as induction and maintenance therapy for ulcerative colitis. N Engl J Med 2019; 381:1201-14 or histologic improvement as defined in Journal of Crohn's and Colitis, Volume 13, Issue 8, August 2019, Pages 1025-103, each of which is incorporated herein by reference in its entirety. Histologic healing is assessed based on the Geboes score (Geboes K, Riddell R, Ost A et al. A reproducible grading scale for histological assessment of inflammation in ulcerative colitis. Gut 2000; 47: 404-9) and is defined as 0-<5% neutrophils in epithelium and no crypt destruction, erosions, ulcerations or granulations (see N Engl J Med 2019; 381:1201-14 and supplementary materials therefor which are incorporated herein by reference in its entirety).

The term “digestive tract” is understood to include the mouth, pharynx, esophagus, stomach, small intestine or small bowel (duodenum, jejunum, ileum), large intestine (colon (cecum, ascending colon, transverse colon, descending colon, sigmoid colon), rectum) and anus.

The term “oral cavity” is understood to include the mouth, the pharynx and the esophagus.

The term “gastrointestinal tract”, or “GI tract” is understood to include the stomach, small intestine or small bowel (duodenum, jejunum, ileum), large intestine (cecum, colon (cecum, ascending colon, transverse colon, descending colon, sigmoid colon), rectum) and anus.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity (e.g., a compound exhibiting activity as a mitochondrial uncoupling agent or a pharmaceutically acceptable salt and/or hydrate and/or cocrystal thereof, e.g., a compound, such as niclosamide or a pharmaceutically acceptable salt and/or hydrate and/or cocrystal thereof, e.g., a compound, such as a niclosamide analog, or a pharmaceutically acceptable salt and/or hydrate and/or cocrystal thereof) being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed; Lippincott Williams & Wilkins: Philadelphia, Pa., 2005; Handbook of Pharmaceutical Excipients, 6th ed; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed; Gibson Ed.; CRC Press LLC: Boca Raton, Fla., 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

The details of one or more embodiments of the invention are set forth in the accompanying drawings and the description below. Other features and advantages of the invention will be apparent from the description and drawings, and from the claims.

### DESCRIPTION OF DRAWINGS

FIG. 1 is a schematic representation of the design of a Phase 1/2a study of Niclosamide Enema in the treatment of subjects with mild-to-moderate UP or UPS with Inadequate Response to 5-ASA.

FIG. 2 is a plot showing the Geboes Score of subjects with mild-to-moderate UP or UPS achieving clinical emission after treatment with Niclosamide Enema (150 mg/60 mL per the rectum for 6 weeks) relative to baseline FIG. 3 is a heat-map of the expression level of cytokines in subjects treated with Niclosamide Enema (150 mg/60 mL per the rectum for 6 weeks) compared to the expression level of these subjects at the baseline.

FIG. 4 is a histogram showing the changes in MMS of subjects treated with Niclosamide Enema (150 mg/60 mL per the rectum for 6 weeks).

FIGS. 5A-5C show the components of a representative enema delivery device (FIG. 5A shows the bottle, FIG. 5B shows the breakable capsule, and FIG. 5C shows the rectal cannula (upper arrow) and single flow pack (lower arrow).

### DETAILED DESCRIPTION

This disclosure features methods for treating one or more conditions (or one or more symptoms thereof) characterized by an abnormal inflammatory response in one or more particular subject (e.g., patient) populations in need thereof. Such conditions include, e.g., an autoimmune disorder, e.g., colitis, e.g., autoimmune colitis, e.g, an inflammatory bowel disease, e.g., Crohn's disease, ulcerative colitis). The methods include administering to the subject an effective amount of niclosamide or a pharmaceutically acceptable salt and/or cocrystal thereof as well as compositions containing the same. In some embodiments, the methods include rectally (e.g., via enema) administering niclosamide.

In certain embodiments, the chemical entity is niclosamide or a pharmaceutically acceptable salt or hydrate thereof “Niclosamide” refers to a compound having the following chemical structure:

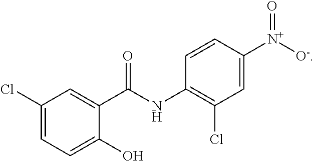

Niclosamide or a pharmaceutically acceptable salt or hydrate or co-crystal thereof and compositions comprising niclosamide or a pharmaceutically acceptable salt or hydrate or co-crystal thereof are disclosed in U.S. Pat. No. 10,292,951, incorporated by reference herein in its entirety.

Niclosamide is known by the IUPAC designation: 2′5-dichloro-4-nitrosalicylanilide and by the CAS designation: CAS: 5-chloro-N-(2-chloro-4-nitrophenyl)-2-hydroxybenzamide. Niclosamide has a relatively low water solubility at about from 5-8 mg/L at 20° C., is sparingly soluble in ether, ethanol and chloroform, and is soluble in acetone. The ethanolamine salt dissolves in distilled water 180-280 mg/L at 20° C.

Niclosamide is available in a various salt or solvated forms. These include, but are not limited to, the ethanolamine salt known by the IUPAC designation 5-chloro-salicyl-(2-chloro-4-nitro) anilide 2-aminoethanol salt or the CAS designation 5-chloro-N-(2-chloro-4-nitrophenyl)-2-hydroxybenzamide with 2-aminoethanol (1:1)—see, e.g., U.S. 2013/0231312; the piperazine salt known by the IUPAC designation 5-chloro-salicyl-(2-chloro-4-nitro) anilide piperazine salt or the CAS designation 5-chloro-N-(2-chloro-4-nitrophenyl)-2-hydroxybenzamide with piperazine (2:1); and niclosamide monohydrate known by the IUPAC designation 5-chloro-salicyl-(2-chloro-4-nitro) anilide monohydrate or the CAS designation 5-chloro-N-(2-chloro-4-nitrophenyl)-2-hydroxybenzamide with monohydrate (1:1).

Niclosamide is commercially available in a variety of formulations including, but not limited to BAYER 73, BAYER 2353®, BAYER 25 648®, BAYLUSCID®, BAYLUSCIDE®, CESTOCID®, CLONITRALID, DICHLOSALE®, FENASA®, HL 2447®, IOMESAN®, IOMEZAN®, LINTEX®, MANOSIL®, NASEMO®, NICLOSAMID®, PHENASAL®, TREDEMINE®, SULQUI®, VERMITID®, VERMITIN®, YOMESAN®, and the like.

Pharmaceutical Compositions and Administration

General

A chemical entity (e.g., a compound exhibiting activity as a mitochondrial uncoupling agent or a pharmaceutically acceptable salt and/or hydrate and/or cocrystal thereof; e.g., a compound, such as niclosamide or a pharmaceutically acceptable salt and/or hydrate and/or cocrystal thereof; e.g., a compound, such as a niclosamide analog, or a pharmaceutically acceptable salt and/or hydrate and/or cocrystal thereof) is administered to a subject in need thereof by any route which makes the compound bioavailable (e.g., locally bioavailable).

In some embodiments, a chemical entity (e.g., a compound exhibiting activity as a mitochondrial uncoupling agent or a pharmaceutically acceptable salt and/or hydrate and/or cocrystal thereof, e.g., a compound, such as niclosamide or a pharmaceutically acceptable salt and/or hydrate and/or cocrystal thereof, e.g., a compound, such as a niclosamide analog, or a pharmaceutically acceptable salt and/or hydrate and/or cocrystal thereof) is administered as a pharmaceutical composition that includes the chemical entity and one or more pharmaceutically acceptable excipients, and optionally one or more other therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumor, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Local Administration

In some embodiments, the chemical entities described herein or a pharmaceutical composition thereof are suitable for local administration, e.g., local administration by way of topically administering the chemical entity or composition thereof at a particular treatment site, (e.g., the digestive tract, the gastrointestinal (“GI”) tract, eye, joint, or skin) so as to provide local administration of the chemical entity to the area in need of treatment (e.g., oral cavity; GI tract, e.g., the colon; eye; skin; or joint). In certain embodiments, minimal systemic exposure of the chemical entity occurs during said local administration. Examples of such compositions include, without limitation, compositions for rectal administration, oral administration, dermal administration, or implant. In certain embodiments, compositions are for other than oral administration.

In some embodiments, the chemical entities described herein or a pharmaceutical composition thereof are suitable for local administration to the GI tract. In certain embodiments, upon administration, the local concentration of the chemical entity in the GI tract is higher (e.g., from about 2 times higher to about 50 times higher, from about 5 times higher to about 50 times higher; from about 5 times higher to about 25 times higher; from about 5 times higher to about 15 times higher; e.g., about 50 times higher, about 25 time higher, about 20 times higher, about 15 times higher, about 10 times higher, about 5 times higher, e.g., at least about 10 times higher) than the concentration of the chemical entity in the plasma compartment. In certain of these embodiments, the chemical entity in the plasma compartment is subject to first pass metabolism.

In some embodiments, the chemical entities described herein or a pharmaceutical composition thereof are suitable for local administration to one or more specific locations within the digestive or GI tract. For example, at least some of the chemical entity is present in the upper GI tract (e.g., stomach); or at least some of the agent is present in the lower GI tract (e.g., the large intestine, e.g., the colon, e.g., the ascending colon and/or transverse colon and/or distal colon; or the small bowel). As a further example, at least some of the chemical entity is present in the ascending colon and/or the transverse colon and/or the distal colon and/or the small bowel and/or the stomach. Methods of said local administration can include, without limitation, rectal administration and/or oral administration.

In certain embodiments, the chemical entities described herein or a pharmaceutical composition thereof are suitable for local, topical administration to the digestive or GI tract, e.g., rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

Enema Formulations

In some embodiments, enema formulations containing the chemical entities described herein are provided in “ready-to-use” form.

In some embodiments, enema formulations containing the chemical entities described herein are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g. natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

In some embodiments, each of the one or more pharmaceutically acceptable excipients can be independently selected from thickeners, viscosity enhancing agents, bulking agents, mucoadhesive agents, penetration enhancers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently selected from thickeners, viscosity enhancing agents, bulking agents, mucoadhesive agents, buffers, preservatives, and fillers.

In certain embodiments, each of the one or more pharmaceutically acceptable excipients can be independently selected from diluents, binders, lubricants, glidants, and disintegrants.

Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C1-C4 alkyl. In certain embodiments, the preservative is methyl 4-hydroxybenzoate(methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phosphate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as cross-linked PVP (Polyplasdone XL from GAF Chemical Corp). In certain embodiments, the disintegrant is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dextrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing the chemical entities described herein include water and one or more (e.g., all) of the following excipients:
One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
One or more (e.g., one or two; e.g., two) preservatives, such as a paraben, e.g., methyl 4-hydroxybenzoate(methylparaben), or a pharmaceutically acceptable salt or ester thereof, propyl 4-hydroxybenzoate (propylparaben), or a pharmaceutically acceptable salt or ester thereof, or a combination thereof;
One or more (e.g., one or two; e.g., two) buffers, such as phosphate buffer system (e.g., sodium dihydrogen phosphate dehydrate, disodium phosphate dodecahydrate);
One or more (e.g., one or two, e.g., two) glidants and/or lubricants, such as magnesium stearate and/or talc;
One or more (e.g., one or two; e.g., one) disintegrants, such as crospovidone; and
One or more (e.g., one or two; e.g., one) diluents, such as lactose (e.g., lactose monohydrate).

In certain of these embodiments, the chemical entity is niclosamide, or a pharmaceutically acceptable salt and/or hydrate and/or cocrystal thereof; e.g., niclosamide.

In certain embodiments, enema formulations containing the chemical entities described herein include water, methyl cellulose, povidone, methylparaben, propylparaben, sodium dihydrogen phosphate dehydrate, disodium phosphate dodecahydrate, crospovidone, lactose monohydrate, magnesium stearate, and talc. In certain of these embodiments, the chemical entity is niclosamide, or a pharmaceutically acceptable salt and/or hydrate and/or cocrystal thereof; e.g., niclosamide.

In certain embodiments, enema formulations containing the chemical entities described herein are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (ii) is provided in its own separate kit or pack.

In certain of these embodiments, component (i) includes the chemical entity (e.g., niclosamide, or a pharmaceutically acceptable salt and/or hydrate and/or cocrystal thereof; e.g., niclosamide) and one or more (e.g., all) of the following excipients:
(a) One or more (e.g., one) binders (e.g., a polyvinyl polymer, such as polyvinylpyrrolidone (povidone);
(b) One or more (e.g., one or two, e.g., two) glidants and/or lubricants, such as magnesium stearate and/or talc;
(c) One or more (e.g., one or two; e.g., one) disintegrants, such as crospovidone; and
(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the chemical entity (e.g., niclosamide, or a pharmaceutically acceptable salt and/or hydrate and/or cocrystal thereof; e.g., niclosamide).

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about 1 weight percent; from about 0.1 weight percent to about 1 weight percent; from about 0.1 weight percent to about 0.5 weight percent; e.g., about 0.27 weight percent) of the lubricant (e.g., magnesium stearate).

In certain embodiments (when component (i) includes one or more lubricants, such as talc), component (i) includes from about 0.5 weight percent to about 5 weight percent (e.g., from about 0.5 weight percent to about 3 weight percent, from about 1 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

In certain embodiments, component (i) includes the ingredients and amounts as shown in Table 7.

TABLE 7

| Ingredient | Weight Percent |
|---|---|
| niclosamide | 40 weight percent to about 80 weight |
| | percent (e.g., from about 50 weight percent |
| | to about 70 weight percent, from about 55 |
| | weight percent to about 70 weight percent; |
| | from about 60 weight percent to about 65 |
| | weight percent; e.g., about 62.1 weight |
| | percent) |
| Crospovidone | 0.5 weight percent to about 5 weight |
| (Kollidon CL) | percent (e.g., from about 0.5 weight |
| | percent to about 3 weight percent, from |
| | about 1 weight percent to about 3 weight |
| | percent; about 1.93 weight percent |
| lactose monohydrate | about 10 weight percent to about 50 weight |
| (Pharmatose 200M) | percent (e.g., from about 20 weight percent |
| | to about 40 weight percent, from about 25 |
| | weight percent to about 35 weight percent; |
| | e.g., about 31.03 weight percent |
| Povidone | about 0.5 weight percent to about 5 weight |
| (Kollidon K30) | percent (e.g., from about 1.5 weight |
| | percent to about 4.5 weight percent, from |
| | about 2 weight percent to about 3.5 weight |
| | percent; e.g., about 2.76 weight percent |
| talc | 0.5 weight percent to about 5 weight |
| | percent (e.g., from about 0.5 weight |
| | percent to about 3 weight percent, from |
| | about 1 weight percent to about 3 weight |
| | percent; from about 1.5 weight percent to |
| | about 2.5 weight percent; from about 1.8 |
| | weight percent to about 2.2 weight |
| | percent; e.g., about 1.93 weight percent |
| Magnesium stearate | about 0.05 weight percent to about 1 |
| | weight percent (e.g., from about 0.05 |
| | weight percent to about 1 weight percent; |
| | from about 0.1 weight percent to about 1 |
| | weight percent; from about 0.1 weight |
| | percent to about 0.5 weight percent; e.g., |
| | about 0.27 weight percent |

In certain embodiments, component (i) includes the ingredients an amounts as shown in Table 8.

TABLE 8

| Ingredient | Weight Percent |
|---|---|
| niclosamide | About 62.1 weight percent) |
| Crospovidone (Kollidon CL) | About 1.93 weight percent |
| lactose monohydrate (Pharmatose 200M) | About 31.03 weight percent |
| Povidone (Kollidon K30) | About 2.76 weight percent |
| talc | About 1.93 weight percent |
| Magnesium stearate | About 0.27 weight percent |

In certain embodiments, component (i) is formulated as a wet granulated solid preparation. In certain of these embodiments an internal phase of ingredients (the chemical entity, disintegrant, and diluent) are combined and mixed in a high-shear granulator. A binder (e.g., povidone) is dissolved in water to form a granulating solution. This solution is added to the Inner Phase mixture resulting in the development of granules. While not wishing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging. See, e.g., Example 8.

In certain of the foregoing embodiments, component (ii) includes water and one or more (e.g., all) of the following excipients:
(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
(b′) One or more (e.g., one or two; e.g., two) preservatives, such as a paraben, e.g., methyl 4-hydroxybenzoate(methylparaben), or a pharmaceutically acceptable salt or ester thereof, propyl 4-hydroxybenzoate (propylparaben), or a pharmaceutically acceptable salt or ester thereof, or a combination thereof; and
(c′) One or more (e.g., one or two; e.g., two) buffers, such as phosphate buffer system (e.g., sodium dihydrogen phosphate dihydrate, disodium phosphate dodecahydrate); In certain of the foregoing embodiments, component (ii) includes water and one or
more (e.g., all) of the following excipients:
(a″) a first thickener, viscosity enhancing agent, binder, and/or mucoadhesive agent (e.g., a cellulose or cellulose ester or ether or derivative or salt thereof (e.g., methyl cellulose));
(a″) a second thickener, viscosity enhancing agent, binder, and/or mucoadhesive agent (e.g., a polyvinyl polymer, such as polyvinylpyrrolidone (povidone));
(b″) a first preservative, such as a paraben, e.g., propyl 4-hydroxybenzoate (propylparaben), or a pharmaceutically acceptable salt or ester thereof;
(b″) a second preservative, such as a paraben, e.g., methyl 4-hydroxybenzoate(methylparaben), or a pharmaceutically acceptable salt or ester thereof,
(c″) a first buffer, such as phosphate buffer system (e.g., disodium phosphate dodecahydrate);
(c′″) a second buffer, such as phosphate buffer system (e.g., sodium dihydrogen phosphate dehydrate),

In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent; e.g., about 1.0 weight percent) of (a′″).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b′″).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of (c′″).

In certain of these embodiments, each of (a″)-(c′″) is present.

In certain embodiments, component (ii) includes water (up to 100%) and the ingredients and amounts as shown in Table 9.

TABLE 9

| Ingredient | Weight Percent |
|---|---|
| methyl cellulose | 0.05 weight percent to about 5 weight |
| (Methocel A15C | percent (e.g., from about 0.05 weight |
| premium) | percent to about 3 weight percent, from |
| | about 0.1 weight percent to about 3 weight |
| | percent; e.g., about 1.4 weight percent |
| Povidone (Kollidon K30) | 0.05 weight percent to about 5 weight |
| | percent (e.g., from about 0.05 weight |
| | percent to about 3 weight percent, from |
| | about 0.1 weight percent to about 2 weight |
| | percent; e.g., about 1.0 weight percent |
| propyl 4-hydroxybenzoate | about 0.005 weight percent to about 0.1 |
| | weight percent (e.g., from about 0.005 |
| | weight percent to about 0.05 weight |
| | percent; e.g., about 0.02 weight percent) |
| methyl 4-hydroxybenzoate | about 0.05 weight percent to about 1 |
| | weight percent (e.g., from about 0.05 |
| | weight percent to about 0.5 weight percent; |
| | e.g., about 0.20 weight percent) |
| disodium phosphate | about 0.05 weight percent to about 1 |
| dodecahydrate | weight percent (e.g., from about 0.05 |
| | weight percent to about 0.5 weight percent; |
| | e.g., about 0.15 weight percent) |
| sodium dihydrogen | about 0.005 weight percent to about 0.5 |
| phospahate dihydrate | weight percent (e.g., from about 0.005 |
| | weight percent to about 0.3 weight percent; |
| | e.g., about 0.15 weight percent) |

In certain embodiments, component (ii) includes water (up to 1000%) and the ingredients and amounts as shown in Table 10.

TABLE 10

| Ingredient | Weight Percent |
|---|---|
| methyl cellulose (Methocel A15C | about 1.4 weight percent |
| premium) | |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate dodecahydrate | about 0.15 weight percent |
| sodium dihydrogen phospahate dihydrate | about 0.15 weight percent |

Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

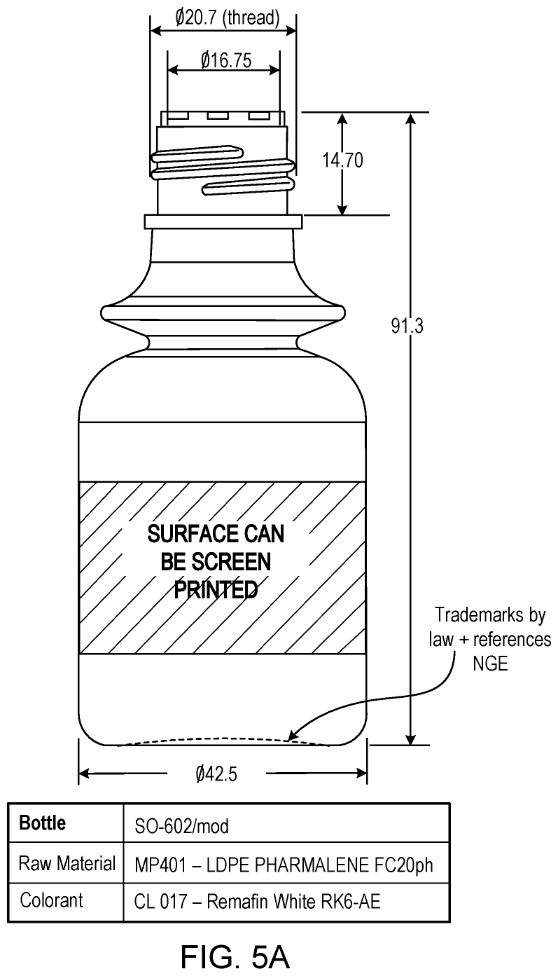

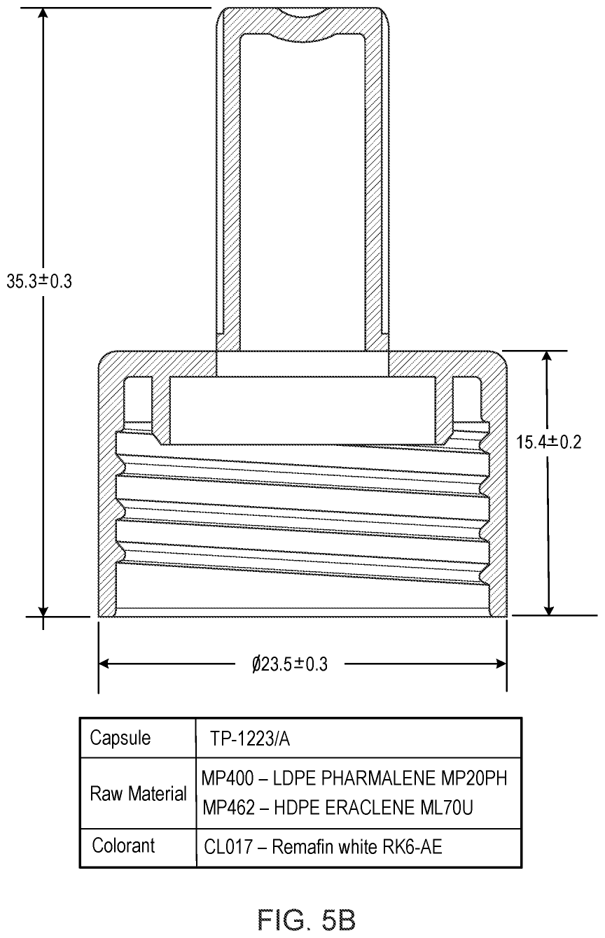

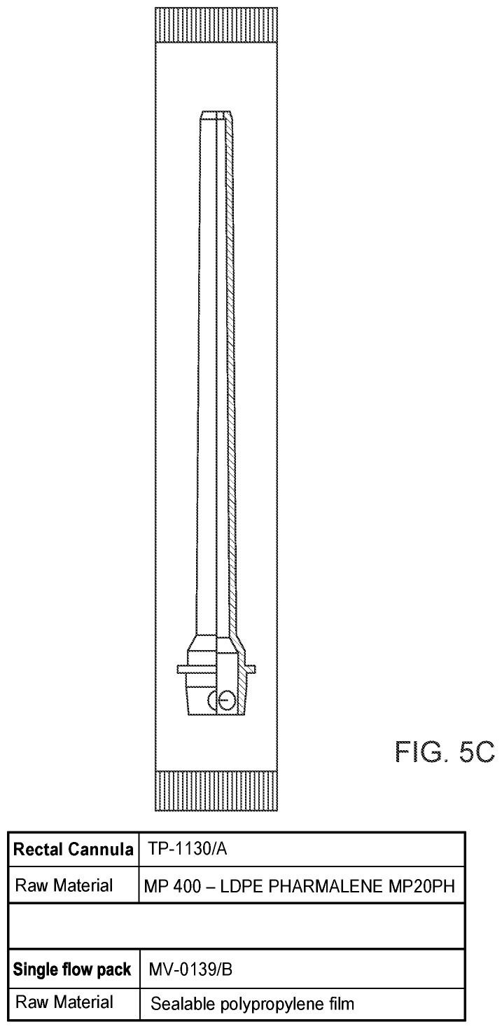

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum. In some embodiments, the enema formulation can be delivered in the device shown in FIGS. 5A-5C, which includes a plastic bottle, a breakable capsule, and a rectal cannula and single flow pack.

Oral Delivery

In other embodiments, the chemical entities described herein or a pharmaceutical composition thereof are suitable for local delivery to the digestive or GI tract by way of oral administration (e.g., solid or liquid dosage forms).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series(methacrylic acid-methyl methacrylate copolymers), and Marcoat). Other techniques include dosage forms that respond to local flora in the GI tract, Pressure-controlled colon delivery capsule, and Pulsincap.

Liquid dosage forms for oral administration include pharmaceutically acceptable emulsions, microemulsions, solutions, suspensions, syrups and elixirs. In addition to the chemical entities described herein, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments, the liquid dosage form is a mouthwash. In certain embodiments, such liquid oral dosage forms are useful for local and topical administration to the digestive or GI tract, e.g., digestive tract, e.g., oral cavity.

Other Forms of Delivery

In some embodiments, the chemical entities described herein or a pharmaceutical composition thereof are suitable for local and topical administration to the eye (e.g., eye drops). Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

In some embodiments, the chemical entities described herein or a pharmaceutical composition thereof are suitable for local and topical administration to skin (e.g., ointments and creams). Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, a chemical entity (e.g., a compound exhibiting activity as a mitochondrial uncoupling agent or a pharmaceutically acceptable salt and/or hydrate and/or cocrystal thereof; e.g., a compound, such as niclosamide or a pharmaceutically acceptable salt and/or hydrate and/or cocrystal thereof; e.g., a compound, such as a niclosamide analog, or a pharmaceutically acceptable salt and/or hydrate and/or cocrystal thereof) is administered is administered at a dosage of from about 0.01 mg/Kg to about 200 mg/Kg (e.g., from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg).

In certain embodiments, the chemical entity is administered at a dosage of from about 15 mg/Kg to about 100 mg/Kg (e.g., from about 15 mg/Kg to about 90 mg/Kg, from about 20 mg/Kg to about 100 mg/Kg; from about 20 mg/Kg to about 90 mg/Kg; from about 20 mg/Kg to about 80 mg/Kg; from about 30 mg/Kg to about 90 mg/Kg; from about 30 mg/Kg to about 80 mg/Kg; from about 35 mg/Kg to about 75 mg/Kg; from about 10 mg/Kg to about 50 mg/Kg; from about 15 mg/Kg to about 45 mg/Kg; e.g., about 35 mg/Kg or about 75 mg/Kg). In other embodiments, the chemical entity is administered at a dosage of from about 0.1 mg/Kg to about 10 mg/Kg (e.g., from about 0.1 mg/Kg to about 5 mg/Kg; from about 1 mg/Kg to about 10 mg/Kg; from about 1 mg/Kg to about 5 mg/Kg).

In some embodiments, enema formulations include from about 0.5 mg to about 2500 mg (e.g., from about 0.5 mg to about 2000 mg, from about 0.5 mg to about 1000 mg, from about 0.5 mg to about 750 mg, from about 0.5 mg to about 600 mg, from about 0.5 mg to about 500 mg, from about 0.5 mg to about 400 mg, from about 0.5 mg to about 300 mg, from about 0.5 mg to about 200 mg; e.g., from about 5 mg to about 2500 mg, from about 5 mg to about 2000 mg, from about 5 mg to about 1000 mg; from about 5 mg to about 750 mg; from about 5 mg to about 600 mg; from about 5 mg to about 500 mg; from about 5 mg to about 400 mg; from about 5 mg to about 300 mg; from about 5 mg to about 200 mg; e.g., from about 50 mg to about 2000 mg, from about 50 mg to about 1000 mg, from about 50 mg to about 750 mg, from about 50 mg to about 600 mg, from about 50 mg to about 500 mg, from about 50 mg to about 400 mg, from about 50 mg to about 300 mg, from about 50 mg to about 200 mg; e.g., from about 100 mg to about 2500 mg, from about 100 mg to about 2000 mg, from about 100 mg to about 1000 mg, from about 100 mg to about 750 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg; e.g., from about 150 mg to about 2500 mg, from about 150 mg to about 2000 mg, from about 150 mg to about 1000 mg, from about 150 mg to about 750 mg, from about 150 mg to about 700 mg, from about 150 mg to about 600 mg, from about 150 mg to about 500 mg, from about 150 mg to about 400 mg, from about 150 mg to about 300 mg, from about 150 mg to about 200 mg; e.g., from about 150 mg to about 500 mg; e.g., from about 300 mg to about 2500 mg, from about 300 mg to about 2000 mg, from about 300 mg to about 1000 mg, from about 300 mg to about 750 mg, from about 300 mg to about 700 mg, from about 300 mg to about 600 mg; e.g., from about 400 mg to about 2500 mg, from about 400 mg to about 2000 mg, from about 400 mg to about 1000 mg, from about 400 mg to about 750 mg, from about 400 mg to about 700 mg, from about 400 mg to about 600 from about 400 mg to about 500 mg; e.g., 150 mg or 450 mg) of the chemical entity in from about 1 mL to about 3000 mL (e.g., from about 1 mL to about 2000 mL, from about 1 mL to about 1000 mL, from about 1 mL to about 500 mL, from about 1 mL to about 250 mL, from about 1 mL to about 100 mL, from about 10 mL to about 1000 mL, from about 10 mL to about 500 mL, from about 10 mL to about 250 mL, from about 10 mL to about 100 mL, from about 30 mL to about 90 mL, from about 40 mL to about 80 mL; from about 50 mL to about 70 mL; e.g., about 1 mL, about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 100 mL, about 250 mL, or about 500 mL, or about 1000 mL, or about 2000 mL, or about 3000 mL; e.g., 60 mL) of liquid carrier.

In certain embodiments, enema formulations include from about 50 mg to about 250 mg (e.g., from about 100 mg to about 200; e.g., about 150 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 150 mg of the chemical entity in about 60 mL of the liquid carrier. In certain of these embodiments, the chemical entity is niclosamide, or a pharmaceutically acceptable salt and/or hydrate and/or cocrystal thereof. For example, enema formulations can include about 150 mg of niclosamide in about 60 mL of the liquid carrier.

In certain embodiments, enema formulations include from about 350 mg to about 550 mg (e.g., from about 400 mg to about 500; e.g., about 450 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 450 mg of the chemical entity in about 60 mL of the liquid carrier. In certain of these embodiments, the chemical entity is niclosamide, or a pharmaceutically acceptable salt and/or hydrate and/or cocrystal thereof. For example, enema formulations can include about 450 mg of niclosamide in about 60 mL of the liquid carrier.

In certain embodiments, enema formulations include from about 800 mg to about 1000 mg (e.g., from about 850 mg to about 950; e.g., about 900 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 900 mg of the chemical entity in about 60 mL of the liquid carrier. In certain of these embodiments, the chemical entity is niclosamide, or a pharmaceutically acceptable salt and/or hydrate and/or cocrystal thereof. For example, enema formulations can include about 900 mg of niclosamide in about 60 mL of the liquid carrier.

In some embodiments, enema formulations include from about from about 0.01 mg/mL to about 50 mg/mL (e.g., from about 0.01 mg/mL to about 25 mg/mL; from about 0.01 mg/mL to about 10 mg/mL; from about 0.01 mg/mL to about 5 mg/mL; from about 0.1 mg/mL to about 50 mg/mL; from about 0.01 mg/mL to about 25 mg/mL; from about 0.1 mg/mL to about 10 mg/mL; from about 0.1 mg/mL to about 5 mg/mL; from about 1 mg/mL to about 10 mg/mL; from about 1 mg/mL to about 5 mg/mL; from about 5 mg/mL to about 10 mg/mL; e.g., about 2.5 mg/mL or about 7.5 mg/mL) of the chemical entity in liquid carrier. In certain of these embodiments, the chemical entity is niclosamide, or a pharmaceutically acceptable salt and/or hydrate and/or cocrystal thereof. For example, enema formulations can include about 2.5 mg/mL or about 7.5 mg/mL of niclosamide in liquid carrier.

The foregoing dosages can be administered on a daily basis (e.g., as a single dose per day; or as two or more divided doses per day; or a two or more doses; e.g., two doses per day) or non-daily basis (e.g., every other day, every two days, every three days, once weekly, twice weeks, once every two weeks, once a month). In certain embodiments, dosages can be administered for about 1 week, about 2 weeks, about 3 weeks, about 4 weeks, about 5 weeks, about 6 weeks, about 7 weeks, about 8 weeks, about 3 months, about 6 months, about 1 year, or beyond. For example, dosages (e.g., about 2.5 mg/mL or about 7.5 mg/mL) of the chemical entity in liquid carrier can be administered twice a day on a daily basis for about 6 weeks. In certain of these embodiments, the chemical entity is niclosamide, or a pharmaceutically acceptable salt and/or hydrate and/or cocrystal thereof. For example, about 2.5 mg/mL or about 7.5 mg/mL of niclosamide in liquid carrier can be administered twice a day on a daily basis for about 6 weeks. Representative liquid carriers include, e.g., those previously described in conjunction with component (ii).

Methods of Treatment

In certain embodiments, provided herein is a method for treating colitis in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject is a subject previously diagnosed with ulcerative proctitis and/or proctosigmoiditis.

In some embodiments the subject has been diagnosed with ulcerative proctitis and/or proctosigmoiditis at least three months prior to said administering.

In some more particular embodiments, the subject has been diagnosed with ulcerative proctitis and/or proctosigmoiditis at least three months prior to said administering, wherein:
a) the subject has a modified Mayo score (MMS) equal to from 4 points or greater than 4 points to 8 points or less than 8 points prior to said administering;
b) the subject has been treated with an aminosalicylate drug prior to said administering.
c) the subject has a modified Mayo score (MMS) equal to less than 2 points following said administering.
d) the subject has at least one of:
   I) a modified Mayo score (MMS) following said administering that is at least 2 points lower than a modified Mayo score (MMS) in the subject prior to said administering;
   II) a modified Mayo score (MMS) following said administering that is at least 25% lower than a modified Mayo score (MMS) in the subject prior to said administering;
   III) a Rectal Bleeding Score (RBS) following said administering that is at least 1 point lower than a Rectal Bleeding Score (RBS) in the subject prior to said administering; or
   IV) a Rectal Bleeding Score (RBS) following said administering that is 0 or 1;
e) the subject does not have a modified endoscopic subscore of 0 or 1 prior to said administering and the subject has a modified endoscopic subscore of 0 or 1 following said administering;
f) following said administering, the subject shows a histologic improvement of at least 20%, such as at least 30%, such as at least 40%, such as at least 50%, relative to prior to said administering;
g) following said administering, the subject shows a decrease in histologic score of at least 10%, such as at least 15%, such as at least 20%, such as at least 30%, such as at least 40%, relative to prior to said administering.
h) following said administering, the subject shows an endoscopic mucosal healing of at least 20%, such as at least 30%, such as at least 40%, relative to prior to said administering;
i) following said administering, the subject shows a decrease in the level of an inflammatory marker relative to the level of the inflammatory marker prior to said administering; and/or
j) following said administering, the subject shows an increase in the level of an inflammatory a marker relative to the level the inflammatory marker prior to said administering;
provided that the inflammatory marker in i) and the inflammatory marker in j) are not the same.

In certain embodiments, provided herein is a method for treating colitis in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has a modified Mayo score (MMS) equal to from 4 points or greater than 4 points to 8 points or less than 8 points prior to said administering.

In some more particular embodiments, the subject has a modified Mayo score (MMS) equal to from 4 points or greater than 4 points to 8 points or less than 8 points prior to said administering, wherein:
a) the subject has been treated with an aminosalicylate drug prior to said administering.
b) the subject has a modified Mayo score (MMS) equal to less than 2 points following said administering.
c) the subject has at least one of:
   I) a modified Mayo score (MMS) following said administering that is at least 2 points lower than a modified Mayo score (MMS) in the subject prior to said administering;
   II) a modified Mayo score (MMS) following said administering that is at least 25% lower than a modified Mayo score (MMS) in the subject prior to said administering;
   III) a Rectal Bleeding Score (RBS) following said administering that is at least 1 point lower than a Rectal Bleeding Score (RBS) in the subject prior to said administering; or
   IV) a Rectal Bleeding Score (RBS) following said administering that is 0 or 1.
d) the subject does not have a modified endoscopic subscore of 0 or 1 prior to said administering and the subject has a modified endoscopic subscore of 0 or 1 following said administering;
e) following said administering, the subject shows a histologic improvement of at least 20%, such as at least 30%, such as at least 40%, such as at least 50%, relative to prior to said administering;
f) following said administering, the subject shows a decrease in histologic score of at least 10%, such as at least 15%, such as at least 20%, such as at least 30%, such as at least 40%, relative to prior to said administering.
g) following said administering, the subject shows an endoscopic mucosal healing of at least 20%, such as at least 30%, such as at least 40%, relative to prior to said administering;
h) following said administering, the subject shows a decrease in the level of an inflammatory marker relative to the level of the inflammatory marker prior to said administering; and/or
i) following said administering, the subject shows an increase in the level of an inflammatory a marker relative to the level the inflammatory marker prior to said administering;
provided that the inflammatory marker in h) and the inflammatory marker in i) are not the same.

In certain embodiments, provided herein is a method for treating colitis in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has been treated with an aminosalicylate drug prior to said administering.

In some more particular embodiments, the subject has been treated with an aminosalicylate drug prior to said administering, wherein:
a) the subject has a modified Mayo score (MMS) equal to less than 2 points following said administering.
b) the subject has at least one of:
   I) a modified Mayo score (MMS) following said administering that is at least 2 points lower than a modified Mayo score (MMS) in the subject prior to said administering;
   II) a modified Mayo score (MMS) following said administering that is at least 25% lower than a modified Mayo score (MMS) in the subject prior to said administering;
   III) a Rectal Bleeding Score (RBS) following said administering that is at least 1 point lower than a Rectal Bleeding Score (RBS) in the subject prior to said administering; or
   IV) a Rectal Bleeding Score (RBS) following said administering that is 0 or 1;
c) the subject does not have a modified endoscopic subscore of 0 or 1 prior to said administering and the subject has a modified endoscopic subscore of 0 or 1 following said administering;
d) following said administering, the subject shows a histologic improvement of at least 20%, such as at least 30%, such as at least 40%, such as at least 50%, relative to prior to said administering;
e) following said administering, the subject shows a decrease in histologic score of at least 10%, such as at least 15%, such as at least 20%, such as at least 30%, such as at least 40%, relative to prior to said administering.
f) following said administering, the subject shows an endoscopic mucosal healing of at least 20%, such as at least 30%, such as at least 40%, relative to prior to said administering;
g) following said administering, the subject shows a decrease in the level of an inflammatory marker relative to the level of the inflammatory marker prior to said administering; and/or
h) following said administering, the subject shows an increase in the level of an inflammatory a marker relative to the level the inflammatory marker prior to said administering;
provided that the inflammatory marker in g) and the inflammatory marker in h) are not the same.

In certain embodiments, provided herein is a method for treating colitis in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has a modified Mayo score (MMS) equal to less than 2 points following said administering.

In some more particular embodiments, the subject has a modified Mayo score (MMS) equal to less than 2 points following said administering, wherein:
a) the subject has at least one of:
   I) a modified Mayo score (MMS) following said administering that is at least 2 points lower than a modified Mayo score (MMS) in the subject prior to said administering;
   II) a modified Mayo score (MMS) following said administering that is at least 25% lower than a modified Mayo score (MMS) in the subject prior to said administering;
   III) a Rectal Bleeding Score (RBS) following said administering that is at least 1 point lower than a Rectal Bleeding Score (RBS) in the subject prior to said administering; or
   IV) a Rectal Bleeding Score (RBS) following said administering that is 0 or 1;
b) the subject does not have a modified endoscopic subscore of 0 or 1 prior to said administering and the subject has a modified endoscopic subscore of 0 or 1 following said administering;
c) following said administering, the subject shows a histologic improvement of at least 20%, such as at least 30%, such as at least 40%, such as at least 50%, relative to prior to said administering;
d) following said administering, the subject shows a decrease in histologic score of at least 10%, such as at least 15%, such as at least 20%, such as at least 30%, such as at least 40%, relative to prior to said administering.
e) following said administering, the subject shows an endoscopic mucosal healing of at least 20%, such as at least 30%, such as at least 40%, relative to prior to said administering;
f) following said administering, the subject shows a decrease in the level of an inflammatory marker relative to the level of the inflammatory marker prior to said administering; and/or
g) following said administering, the subject shows an increase in the level of an inflammatory a marker relative to the level the inflammatory marker prior to said administering;

provided that the inflammatory marker in f) and the inflammatory marker in g) are not the same.

In certain embodiments, provided herein is a method for treating colitis in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier, wherein the subject has at least one of:
(I) a modified Mayo score (MMS) following said administering that is at least 2 points lower than a modified Mayo score (MMS) in the subject prior to said administering;
(II) a modified Mayo score (MMS) following said administering that is at least 25% lower than a modified Mayo score (MMS) in the subject prior to said administering;
(III) a Rectal Bleeding Score (RBS) following said administering that is at least 1 point lower than a Rectal Bleeding Score (RBS) in the subject prior to said administering; or
(IV) a Rectal Bleeding Score (RBS) following said administering that is 0 or 1.

In some more particular embodiments, wherein the subject has at least one of (I), (II0, (III) or (IV), wherein:
a) the subject does not have a modified endoscopic subscore of 0 or 1 prior to said administering and the subject has a modified endoscopic subscore of 0 or 1 following said administering;
b) following said administering, the subject shows a histologic improvement of at least 20%, such as at least 30%, such as at least 40%, such as at least 50%, relative to prior to said administering;
c) following said administering, the subject shows a decrease in histologic score of at least 10%, such as at least 15%, such as at least 20%, such as at least 30%, such as at least 40%, relative to prior to said administering.
d) following said administering, the subject shows an endoscopic mucosal healing of at least 20%, such as at least 30%, such as at least 40%, relative to prior to said administering;
e) following said administering, the subject shows a decrease in the level of an inflammatory marker relative to the level of the inflammatory marker prior to said administering; and/or
f) following said administering, the subject shows an increase in the level of an inflammatory a marker relative to the level the inflammatory marker prior to said administering;

provided that the inflammatory marker in e) and the inflammatory marker in f) are not the same.

In some embodiments, provided herein is a method for treating colitis in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject does not have a modified endoscopic subscore of 0 or 1 prior to said administering and the subject has a modified endoscopic subscore of 0 or 1 following said administering.

In some more particular embodiments, the subject does not have a modified endoscopic subscore of 0 or 1 prior to said administering and the subject has a modified endoscopic subscore of 0 or 1 following said administering, wherein:
a) following said administering, the subject shows a histologic improvement of at least 20%, such as at least 30%, such as at least 40%, such as at least 50%, relative to prior to said administering;
b) following said administering, the subject shows a decrease in histologic score of at least 10%, such as at least 15%, such as at least 20%, such as at least 30%, such as at least 40%, relative to prior to said administering.
c) following said administering, the subject shows an endoscopic mucosal healing of at least 20%, such as at least 30%, such as at least 40%, relative to prior to said administering;
d) following said administering, the subject shows a decrease in the level of an inflammatory marker relative to the level of the inflammatory marker prior to said administering; and/or
e) following said administering, the subject shows an increase in the level of an inflammatory a marker relative to the level the inflammatory marker prior to said administering;

provided that the inflammatory marker in d) and the inflammatory marker in e) are not the same.

In some embodiments provided herein is a method for treating colitis in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows a histologic improvement of at least 20%, such as at least 30%, such as at least 40%, such as at least 50%, relative to prior to said administering.

In some more particular embodiments, the subject shows a histologic improvement of at least 20%, such as at least 30%, such as at least 40%, such as at least 50%, relative to prior to said administering, wherein:
a) following said administering, the subject shows a decrease in histologic score of at least 10%, such as at least 15%, such as at least 20%, such as at least 30%, such as at least 40%, relative to prior to said administering.
b) following said administering, the subject shows an endoscopic mucosal healing of at least 20%, such as at least 30%, such as at least 40%, relative to prior to said administering;
c) following said administering, the subject shows a decrease in the level of an inflammatory marker relative to the level of the inflammatory marker prior to said administering; and/or
d) following said administering, the subject shows an increase in the level of an inflammatory a marker relative to the level the inflammatory marker prior to said administering;

provided that the inflammatory marker in c) and the inflammatory marker in d) are not the same.

In some embodiments provided herein is a method for treating colitis in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows a decrease in histologic score of at least 10%, such as at least 15%, such as at least 20%, such as at least 30%, such as at least 40%, relative to prior to said administering.

In some more particular embodiments, the subject shows a decrease in histologic score of at least 10%, such as at least 15%, such as at least 20%, such as at least 30%, such as at least 40%, relative to prior to said administering, wherein:
a) following said administering, the subject shows an endoscopic mucosal healing of at least 20%, such as at least 30%, such as at least 40%, relative to prior to said administering;
b) following said administering, the subject shows a decrease in the level of an inflammatory marker relative to the level of the inflammatory marker prior to said administering; and/or
c) following said administering, the subject shows an increase in the level of an inflammatory a marker relative to the level the inflammatory marker prior to said administering;

provided that the inflammatory marker in b) and the inflammatory marker in c) are not the same.

In some embodiments provided herein is a method for treating colitis in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows an endoscopic mucosal healing of at least 20%, such as at least 30%, such as at least 40%, relative to prior to said administering.

In some more particular embodiments, the subject shows a histologic improvement of at least 20%, such as at least 30%, such as at least 40%, such as at least 50%, relative to prior to said administering, wherein:
a) following said administering, the subject shows a decrease in the level of an inflammatory marker relative to the level of the inflammatory marker prior to said administering; and/or
b) following said administering, the subject shows an increase in the level of an inflammatory a marker relative to the level the inflammatory marker prior to said administering;

provided that the inflammatory marker in a) and the inflammatory marker in b) are not the same.

In some embodiments provided herein is a method for treating colitis in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows a decrease in the level of an inflammatory marker relative to the level of the inflammatory marker prior to said administering.

In some more particular embodiments, the level of the inflammatory marker is the level of the marker in colonic mucosa. In some more particular embodiments, the level of the inflammatory marker is the level of the marker in plasma. In some more particular embodiments, the inflammatory marker is TNFα. In some more particular embodiments, the inflammatory marker is IL-12. In some more particular embodiments, the decrease in the level of the inflammatory marker following said administering relative to the level of the inflammatory marker prior to said administering is of at least 5%, such as at least 10%, such as at least 15%, such as at least 20%, such as at least 30%, such as at least 40%, such as at least 50%, such as at least 60%, such as at least 70%, such as at least 80%.

In some embodiments provided herein is a method for treating colitis in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows an increase in the level of an inflammatory a marker relative to the level the inflammatory marker prior to said administering.

In certain embodiments, provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject is a subject previously diagnosed with ulcerative proctitis and/or proctosigmoiditis.

In some embodiments the subject has been diagnosed with ulcerative proctitis and/or proctosigmoiditis at least three months prior to said administering.

In some more particular embodiments, the subject has been diagnosed with ulcerative proctitis and/or proctosigmoiditis at least three months prior to said administering, wherein:
a) the subject has a modified Mayo score (MMS) equal to from 4 points or greater than 4 points to 8 points or less than 8 points prior to said administering;
b) the subject has been treated with an aminosalicylate drug prior to said administering.
c) the subject has a modified Mayo score (MMS) equal to less than 2 points following said administering.
d) the subject has at least one of:
   I) a modified Mayo score (MMS) following said administering that is at least 2 points lower than a modified Mayo score (MMS) in the subject prior to said administering;
   II) a modified Mayo score (MMS) following said administering that is at least 25% lower than a modified Mayo score (MMS) in the subject prior to said administering;
   III) a Rectal Bleeding Score (RBS) following said administering that is at least 1 point lower than a Rectal Bleeding Score (RBS) in the subject prior to said administering; or
   IV) a Rectal Bleeding Score (RBS) following said administering that is 0 or 1;
e) the subject does not have a modified endoscopic subscore of 0 or 1 prior to said administering and the subject has a modified endoscopic subscore of 0 or 1 following said administering;
f) following said administering, the subject shows a histologic improvement of at least 20%, such as at least 30%, such as at least 40%, such as at least 50%, relative to prior to said administering;
g) following said administering, the subject shows a decrease in histologic score of at least 10%, such as at least 15%, such as at least 20%, such as at least 30%, such as at least 40%, relative to prior to said administering.
h) following said administering, the subject shows an endoscopic mucosal healing of at least 20%, such as at least 30%, such as at least 40%, relative to prior to said administering;
i) following said administering, the subject shows a decrease in the level of an inflammatory marker relative to the level of the inflammatory marker prior to said administering; and/or
j) following said administering, the subject shows an increase in the level of an inflammatory a marker relative to the level the inflammatory marker prior to said administering;
provided that the inflammatory marker in i) and the inflammatory marker in j) are not the same.

In certain embodiments, provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has a modified Mayo score (MMS) equal to from 4 points or greater than 4 points to 8 points or less than 8 points prior to said administering.

In some more particular embodiments, the subject has a modified Mayo score (MMS) equal to from 4 points or greater than 4 points to 8 points or less than 8 points prior to said administering, wherein:
a) the subject has been treated with an aminosalicylate drug prior to said administering.
b) the subject has a modified Mayo score (MMS) equal to less than 2 points following said administering.
c) the subject has at least one of:
   I) a modified Mayo score (MMS) following said administering that is at least 2 points lower than a modified Mayo score (MMS) in the subject prior to said administering;
   II) a modified Mayo score (MMS) following said administering that is at least 25% lower than a modified Mayo score (MMS) in the subject prior to said administering;
   III) a Rectal Bleeding Score (RBS) following said administering that is at least 1 point lower than a Rectal Bleeding Score (RBS) in the subject prior to said administering; or
   IV) a Rectal Bleeding Score (RBS) following said administering that is 0 or 1;
d) the subject does not have a modified endoscopic subscore of 0 or 1 prior to said administering and the subject has a modified endoscopic subscore of 0 or 1 following said administering;
e) following said administering, the subject shows a histologic improvement of at least 20%, such as at least 30%, such as at least 40%, such as at least 50%, relative to prior to said administering;
f) following said administering, the subject shows a decrease in histologic score of at least 10%, such as at least 15%, such as at least 20%, such as at least 30%, such as at least 40%, relative to prior to said administering.
g) following said administering, the subject shows an endoscopic mucosal healing of at least 20%, such as at least 30%, such as at least 40%, relative to prior to said administering;
h) following said administering, the subject shows a decrease in the level of an inflammatory marker relative to the level of the inflammatory marker prior to said administering; and/or
i) following said administering, the subject shows an increase in the level of an inflammatory a marker relative to the level the inflammatory marker prior to said administering;
provided that the inflammatory marker in h) and the inflammatory marker in i) are not the same.

In certain embodiments, provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has been treated with an aminosalicylate drug prior to said administering.

In some more particular embodiments, the subject has been treated with an aminosalicylate drug prior to said administering, wherein:
a) the subject has a modified Mayo score (MMS) equal to less than 2 points following said administering.
b) the subject has at least one of:
   I) a modified Mayo score (MMS) following said administering that is at least 2 points lower than a modified Mayo score (MMS) in the subject prior to said administering;
   II) a modified Mayo score (MMS) following said administering that is at least 25% lower than a modified Mayo score (MMS) in the subject prior to said administering;
   III) a Rectal Bleeding Score (RBS) following said administering that is at least 1 point lower than a Rectal Bleeding Score (RBS) in the subject prior to said administering; or
   IV) a Rectal Bleeding Score (RBS) following said administering that is 0 or 1;
c) the subject does not have a modified endoscopic subscore of 0 or 1 prior to said administering and the subject has a modified endoscopic subscore of 0 or 1 following said administering;
d) following said administering, the subject shows a histologic improvement of at least 20%, such as at least 30%, such as at least 40%, such as at least 50%, relative to prior to said administering;
e) following said administering, the subject shows a decrease in histologic score of at least 10%, such as at least 15%, such as at least 20%, such as at least 30%, such as at least 40%, relative to prior to said administering.
f) following said administering, the subject shows an endoscopic mucosal healing of at least 20%, such as at least 30%, such as at least 40%, relative to prior to said administering;
g) following said administering, the subject shows a decrease in the level of an inflammatory marker relative to the level of the inflammatory marker prior to said administering; and/or
h) following said administering, the subject shows an increase in the level of an inflammatory a marker relative to the level the inflammatory marker prior to said administering;

provided that the inflammatory marker in g) and the inflammatory marker in h) are not the same.

In certain embodiments, provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has a modified Mayo score (MMS) equal to less than 2 points following said administering.

In some more particular embodiments, the subject has a modified Mayo score (MMS) equal to less than 2 points following said administering, wherein:
a) the subject has at least one of:
   I) a modified Mayo score (MMS) following said administering that is at least 2 points lower than a modified Mayo score (MMS) in the subject prior to said administering;
   II) a modified Mayo score (MMS) following said administering that is at least 25% lower than a modified Mayo score (MMS) in the subject prior to said administering;
   III) a Rectal Bleeding Score (RBS) following said administering that is at least 1 point lower than a Rectal Bleeding Score (RBS) in the subject prior to said administering; or
   IV) a Rectal Bleeding Score (RBS) following said administering that is 0 or 1;
b) the subject does not have a modified endoscopic subscore of 0 or 1 prior to said administering and the subject has a modified endoscopic subscore of 0 or 1 following said administering;
c) following said administering, the subject shows a histologic improvement of at least 20%, such as at least 30%, such as at least 40%, such as at least 50%, relative to prior to said administering;
d) following said administering, the subject shows a decrease in histologic score of at least 10%, such as at least 15%, such as at least 20%, such as at least 30%, such as at least 40%, relative to prior to said administering.
e) following said administering, the subject shows an endoscopic mucosal healing of at least 20%, such as at least 30%, such as at least 40%, relative to prior to said administering;
f) following said administering, the subject shows a decrease in the level of an inflammatory marker relative to the level of the inflammatory marker prior to said administering; and/or
g) following said administering, the subject shows an increase in the level of an inflammatory a marker relative to the level the inflammatory marker prior to said administering;

provided that the inflammatory marker in f) and the inflammatory marker in g) are not the same.

In certain embodiments, provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has at least one of:
I) a modified Mayo score (MMS) following said administering that is at least 2 points lower than a modified Mayo score (MMS) in the subject prior to said administering;
II) a modified Mayo score (MMS) following said administering that is at least 25% lower than a modified Mayo score (MMS) in the subject prior to said administering;
III) a Rectal Bleeding Score (RBS) following said administering that is at least 1 point lower than a Rectal Bleeding Score (RBS) in the subject prior to said administering; or
IV) a Rectal Bleeding Score (RBS) following said administering that is 0 or 1.

In some more particular embodiments, wherein the subject has at least one of (I), (110, (III) or (IV), wherein:
a) the subject does not have a modified endoscopic subscore of 0 or 1 prior to said administering and the subject has a modified endoscopic subscore of 0 or 1 following said administering;
b) following said administering, the subject shows a histologic improvement of at least 20%, such as at least 30%, such as at least 40%, such as at least 50%, relative to prior to said administering;
c) following said administering, the subject shows a decrease in histologic score of at least 10%, such as at least 15%, such as at least 20%, such as at least 30%, such as at least 40%, relative to prior to said administering.
d) following said administering, the subject shows an endoscopic mucosal healing of at least 20%, such as at least 30%, such as at least 40%, relative to prior to said administering;
e) following said administering, the subject shows a decrease in the level of an inflammatory marker relative to the level of the inflammatory marker prior to said administering; and/or
f) following said administering, the subject shows an increase in the level of an inflammatory a marker relative to the level the inflammatory marker prior to said administering;

provided that the inflammatory marker in e) and the inflammatory marker in f) are not the same.

In some embodiments, provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject does not have a modified endoscopic subscore of 0 or 1 prior to said administering and the subject has a modified endoscopic subscore of 0 or 1 following said administering.

In some more particular embodiments, the subject does not have a modified endoscopic subscore of 0 or 1 prior to said administering and the subject has a modified endoscopic subscore of 0 or 1 following said administering, wherein:
a) following said administering, the subject shows a histologic improvement of at least 20%, such as at least 30%, such as at least 40%, such as at least 50%, relative to prior to said administering;
b) following said administering, the subject shows a decrease in histologic score of at least 10%, such as at least 15%, such as at least 20%, such as at least 30%, such as at least 40%, relative to prior to said administering.
c) following said administering, the subject shows an endoscopic mucosal healing of at least 20%, such as at least 30%, such as at least 40%, relative to prior to said administering;
d) following said administering, the subject shows a decrease in the level of an inflammatory marker relative to the level of the inflammatory marker prior to said administering; and/or
e) following said administering, the subject shows an increase in the level of an inflammatory a marker relative to the level the inflammatory marker prior to said administering;

provided that the inflammatory marker in d) and the inflammatory marker in e) are not the same.

In some embodiments provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows a histologic improvement of at least 20%, such as at least 30%, such as at least 40%, such as at least 50%, relative to prior to said administering.

In some more particular embodiments, the subject shows a histologic improvement of at least 20%, such as at least 30%, such as at least 40%, such as at least 50%, relative to prior to said administering, wherein:
a) following said administering, the subject shows a decrease in histologic score of at least 10%, such as at least 15%, such as at least 20%, such as at least 30%, such as at least 40%, relative to prior to said administering.
b) following said administering, the subject shows an endoscopic mucosal healing of at least 20%, such as at least 30%, such as at least 40%, relative to prior to said administering;
c) following said administering, the subject shows a decrease in the level of an inflammatory marker relative to the level of the inflammatory marker prior to said administering; and/or
d) following said administering, the subject shows an increase in the level of an inflammatory a marker relative to the level the inflammatory marker prior to said administering;

provided that the inflammatory marker in c) and the inflammatory marker in d) are not the same.

In some embodiments provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows a decrease in histologic score of at least 10%, such as at least 15%, such as at least 20%, such as at least 30%, such as at least 40%, relative to prior to said administering.

In some more particular embodiments, the subject shows a decrease in histologic score of at least 10%, such as at least 15%, such as at least 20%, such as at least 30%, such as at least 40%, relative to prior to said administering, wherein:
a) following said administering, the subject shows an endoscopic mucosal healing of at least 20%, such as at least 30%, such as at least 40%, relative to prior to said administering;
b) following said administering, the subject shows a decrease in the level of an inflammatory marker relative to the level of the inflammatory marker prior to said administering; and/or
c) following said administering, the subject shows an increase in the level of an inflammatory a marker relative to the level the inflammatory marker prior to said administering;

provided that the inflammatory marker in b) and the inflammatory marker in c) are not the same.

In some embodiments provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows an endoscopic mucosal healing of at least 20%, such as at least 30%, such as at least 40%, relative to prior to said administering.

In some more particular embodiments, the subject shows a histologic improvement of at least 20%, such as at least 30%, such as at least 40%, such as at least 50%, relative to prior to said administering, wherein:
a) following said administering, the subject shows a decrease in the level of an inflammatory marker relative to the level of the inflammatory marker prior to said administering; and/or
b) following said administering, the subject shows an increase in the level of an inflammatory a marker relative to the level the inflammatory marker prior to said administering;

provided that the inflammatory marker in a) and the inflammatory marker in b) are not the same.

In some embodiments provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows a decrease in the level of an inflammatory marker relative to the level of the inflammatory marker prior to said administering.

In some more particular embodiments, the level of the inflammatory marker is the level of the marker in colonic mucosa. In some more particular embodiments, the level of the inflammatory marker is the level of the marker in plasma. In some more particular embodiments, the inflammatory marker is TNFα. In some more particular embodiments, the inflammatory marker is IL-12. In some more particular embodiments, the inflammatory marker is IFNγ. In some more particular embodiments, the inflammatory marker is IL-17. In some more particular embodiments, the inflammatory marker is IL-23. In some more particular embodiments, the inflammatory marker is IL-22. In some more particular embodiments, the inflammatory marker is IL-5. In some more particular embodiments, the inflammatory marker is IL-13. In some more particular embodiments, the inflammatory marker is MMP3. In some more particular embodiments, the decrease in the level of the inflammatory marker following said administering relative to the level of the inflammatory marker prior to said administering is of at least 5%, such as at least 10%, such as at least 15%, such as at least 20%, such as at least 30%, such as at least 40%, such as at least 50%, such as at least 60%, such as at least 70%, such as at least 80%.

In some embodiments provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows an increase in the level of an inflammatory a marker relative to the level the inflammatory marker prior to said administering.

In certain embodiments, provided herein is a method for treating a condition (or one or more symptoms thereof) characterized by an abnormal inflammatory response in a subject in need thereof are provided (e.g., an autoimmune disorder, e.g., an inflammatory bowel disease) in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject is a subject previously diagnosed with ulcerative proctitis and/or proctosigmoiditis.

In some embodiments the subject has been diagnosed with ulcerative proctitis and/or proctosigmoiditis at least three months prior to said administering.

In some more particular embodiments, the subject has been diagnosed with ulcerative proctitis and/or proctosigmoiditis at least three months prior to said administering, wherein
a) the subject has a modified Mayo score (MMS) equal to from 4 points or greater than 4 points to 8 points or less than 8 points prior to said administering;
b) the subject has been treated with an aminosalicylate drug prior to said administering.
c) the subject has a modified Mayo score (MMS) equal to less than 2 points following said administering.
d) the subject has at least one of:
   I) a modified Mayo score (MMS) following said administering that is at least 2 points lower than a modified Mayo score (MMS) in the subject prior to said administering;
   II) a modified Mayo score (MMS) following said administering that is at least 25% lower than a modified Mayo score (MMS) in the subject prior to said administering;
   III) a Rectal Bleeding Score (RBS) following said administering that is at least 1 point lower than a Rectal Bleeding Score (RBS) in the subject prior to said administering; or
   IV) a Rectal Bleeding Score (RBS) following said administering that is 0 or 1;
e) the subject does not have a modified endoscopic subscore of 0 or 1 prior to said administering and the subject has a modified endoscopic subscore of 0 or 1 following said administering;
f) following said administering, the subject shows a histologic improvement of at least 20%, such as at least 30%, such as at least 40%, such as at least 50%, relative to prior to said administering;
g) following said administering, the subject shows a decrease in histologic score of at least 10%, such as at least 15%, such as at least 20%, such as at least 30%, such as at least 40%, relative to prior to said administering.
h) following said administering, the subject shows an endoscopic mucosal healing of at least 20%, such as at least 30%, such as at least 40%, relative to prior to said administering;
i) following said administering, the subject shows a decrease in the level of an inflammatory marker relative to the level of the inflammatory marker prior to said administering;
and/or
j) following said administering, the subject shows an increase in the level of an inflammatory a marker relative to the level the inflammatory marker prior to said administering;
provided that the inflammatory marker in i) and the inflammatory marker in j) are not the same.

In certain embodiments, provided herein is a method for treating a condition (or one or more symptoms thereof) characterized by an abnormal inflammatory response in a subject in need thereof are provided (e.g., an autoimmune disorder, e.g., an inflammatory bowel disease) in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has a modified Mayo score (MMS) equal to from 4 points or greater than 4 points to 8 points or less than 8 points prior to said administering.

In some more particular embodiments, the subject has a modified Mayo score (MMS) equal to from 4 points or greater than 4 points to 8 points or less than 8 points prior to said administering, wherein:
a) the subject has been treated with an aminosalicylate drug prior to said administering.
b) the subject has a modified Mayo score (MMS) equal to less than 2 points following said administering.
c) the subject has at least one of:
   I) a modified Mayo score (MMS) following said administering that is at least 2 points lower than a modified Mayo score (MMS) in the subject prior to said administering;
   II) a modified Mayo score (MMS) following said administering that is at least 25% lower than a modified Mayo score (MMS) in the subject prior to said administering;
   III) a Rectal Bleeding Score (RBS) following said administering that is at least 1 point lower than a Rectal Bleeding Score (RBS) in the subject prior to said administering; or
   IV) a Rectal Bleeding Score (RBS) following said administering that is 0 or 1;
d) the subject does not have a modified endoscopic subscore of 0 or 1 prior to said administering and the subject has a modified endoscopic subscore of 0 or 1 following said administering;
e) following said administering, the subject shows a histologic improvement of at least 20%, such as at least 30%, such as at least 40%, such as at least 50%, relative to prior to said administering;
f) following said administering, the subject shows a decrease in histologic score of at least 10%, such as at least 15%, such as at least 20%, such as at least 30%, such as at least 40%, relative to prior to said administering.
g) following said administering, the subject shows an endoscopic mucosal healing of at least 20%, such as at least 30%, such as at least 40%, relative to prior to said administering;
h) following said administering, the subject shows a decrease in the level of an inflammatory marker relative to the level of the inflammatory marker prior to said administering; and/or
i) following said administering, the subject shows an increase in the level of an inflammatory a marker relative to the level the inflammatory marker prior to said administering;
provided that the inflammatory marker in h) and the inflammatory marker in i) are not the same.

In certain embodiments, provided herein is a method for treating a condition (or one or more symptoms thereof) characterized by an abnormal inflammatory response in a subject in need thereof are provided (e.g., an autoimmune disorder, e.g., an inflammatory bowel disease) in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has been treated with an aminosalicylate drug prior to said administering.

In some more particular embodiments, the subject has been treated with an aminosalicylate drug prior to said administering, wherein:
a) the subject has a modified Mayo score (MMS) equal to less than 2 points following said administering.
b) the subject has at least one of:
   I) a modified Mayo score (MMS) following said administering that is at least 2 points lower than a modified Mayo score (MMS) in the subject prior to said administering;
   II) a modified Mayo score (MMS) following said administering that is at least 25% lower than a modified Mayo score (MMS) in the subject prior to said administering;
   III) a Rectal Bleeding Score (RBS) following said administering that is at least 1 point lower than a Rectal Bleeding Score (RBS) in the subject prior to said administering; or
   IV) a Rectal Bleeding Score (RBS) following said administering that is 0 or 1;
c) the subject does not have a modified endoscopic subscore of 0 or 1 prior to said administering and the subject has a modified endoscopic subscore of 0 or 1 following said administering;
d) following said administering, the subject shows a histologic improvement of at least 20%, such as at least 30%, such as at least 40%, such as at least 50%, relative to prior to said administering;
e) following said administering, the subject shows a decrease in histologic score of at least 10%, such as at least 15%, such as at least 20%, such as at least 30%, such as at least 40%, relative to prior to said administering.
f) following said administering, the subject shows an endoscopic mucosal healing of at least 20%, such as at least 30%, such as at least 40%, relative to prior to said administering;
g) following said administering, the subject shows a decrease in the level of an inflammatory marker relative to the level of the inflammatory marker prior to said administering; and/or
h) following said administering, the subject shows an increase in the level of an inflammatory a marker relative to the level the inflammatory marker prior to said administering;

provided that the inflammatory marker in g) and the inflammatory marker in h) are not the same.

In certain embodiments, provided herein is a method for treating a condition (or one or more symptoms thereof) characterized by an abnormal inflammatory response in a subject in need thereof are provided (e.g., an autoimmune disorder, e.g., an inflammatory bowel disease) in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has a modified Mayo score (MMS) equal to less than 2 points following said administering.

In some more particular embodiments, the subject has a modified Mayo score (MMS) equal to less than 2 points following said administering, wherein:
a) the subject has at least one of:
   I) a modified Mayo score (MMS) following said administering that is at least 2 points lower than a modified Mayo score (MMS) in the subject prior to said administering;
   II) a modified Mayo score (MMS) following said administering that is at least 25% lower than a modified Mayo score (MMS) in the subject prior to said administering;
   III) a Rectal Bleeding Score (RBS) following said administering that is at least 1 point lower than a Rectal Bleeding Score (RBS) in the subject prior to said administering; or
   IV) a Rectal Bleeding Score (RBS) following said administering that is 0 or 1;
b) the subject does not have a modified endoscopic subscore of 0 or 1 prior to said administering and the subject has a modified endoscopic subscore of 0 or 1 following said administering;
c) following said administering, the subject shows a histologic improvement of at least 20%, such as at least 30%, such as at least 40%, such as at least 50%, relative to prior to said administering;
d) following said administering, the subject shows a decrease in histologic score of at least 10%, such as at least 15%, such as at least 20%, such as at least 30%, such as at least 40%, relative to prior to said administering.
e) following said administering, the subject shows an endoscopic mucosal healing of at least 20%, such as at least 30%, such as at least 40%, relative to prior to said administering;
f) following said administering, the subject shows a decrease in the level of an inflammatory marker relative to the level of the inflammatory marker prior to said administering; and/or
g) following said administering, the subject shows an increase in the level of an inflammatory a marker relative to the level the inflammatory marker prior to said administering;

provided that the inflammatory marker in f) and the inflammatory marker in g) are not the same.

In certain embodiments, provided herein is a method for treating a condition (or one or more symptoms thereof) characterized by an abnormal inflammatory response in a subject in need thereof are provided (e.g., an autoimmune disorder, e.g., an inflammatory bowel disease) in a subject in need thereof, the method comprising administering by enema an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has at least one of:
I) a modified Mayo score (MMS) following said administering that is at least 2 points lower than a modified Mayo score (MMS) in the subject prior to said administering;
II) a modified Mayo score (MMS) following said administering that is at least 25% lower than a modified Mayo score (MMS) in the subject prior to said administering;
III) a Rectal Bleeding Score (RBS) following said administering that is at least 1 point lower than a Rectal Bleeding Score (RBS) in the subject prior to said administering; or
IV) a Rectal Bleeding Score (RBS) following said administering that is 0 or 1.

In some more particular embodiments, wherein the subject has at least one of (I), (II), (III) or (IV), wherein:
a) the subject does not have a modified endoscopic subscore of 0 or 1 prior to said administering and the subject has a modified endoscopic subscore of 0 or 1 following said administering;
b) following said administering, the subject shows a histologic improvement of at least 20%, such as at least 30%, such as at least 40%, such as at least 50%, relative to prior to said administering;
c) following said administering, the subject shows a decrease in histologic score of at least 10%, such as at least 15%, such as at least 20%, such as at least 30%, such as at least 40%, relative to prior to said administering.
d) following said administering, the subject shows an endoscopic mucosal healing of at least 20%, such as at least 30%, such as at least 40%, relative to prior to said administering;
e) following said administering, the subject shows a decrease in the level of an inflammatory marker relative to the level of the inflammatory marker prior to said administering; and/or
f) following said administering, the subject shows an increase in the level of an inflammatory a marker relative to the level the inflammatory marker prior to said administering;

provided that the inflammatory marker in e) and the inflammatory marker in f) are not the same.

In some embodiments, provided herein is a method for treating a condition (or one or more symptoms thereof) characterized by an abnormal inflammatory response in a subject in need thereof are provided (e.g., an autoimmune disorder, e.g., an inflammatory bowel disease) in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject does not have a modified endoscopic subscore of 0 or 1 prior to said administering and the subject has a modified endoscopic subscore of 0 or 1 following said administering.

In some more particular embodiments, the subject does not have a modified endoscopic subscore of 0 or 1 prior to said administering and the subject has a modified endoscopic subscore of 0 or 1 following said administering, wherein:
a) following said administering, the subject shows a histologic improvement of at least 20%, such as at least 30%, such as at least 40%, such as at least 50%, relative to prior to said administering;
b) following said administering, the subject shows a decrease in histologic score of at least 10%, such as at least 15%, such as at least 20%, such as at least 30%, such as at least 40%, relative to prior to said administering.
c) following said administering, the subject shows an endoscopic mucosal healing of at least 20%, such as at least 30%, such as at least 40%, relative to prior to said administering;
d) following said administering, the subject shows a decrease in the level of an inflammatory marker relative to the level of the inflammatory marker prior to said administering; and/or
e) following said administering, the subject shows an increase in the level of an inflammatory a marker relative to the level the inflammatory marker prior to said administering;

provided that the inflammatory marker in d) and the inflammatory marker in e) are not the same.

In some embodiments provided herein is a method for treating a condition (or one or more symptoms thereof) characterized by an abnormal inflammatory response in a subject in need thereof are provided (e.g., an autoimmune disorder, e.g., an inflammatory bowel disease) in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows a histologic improvement of at least 20%, such as at least 30%, such as at least 40%, such as at least 50%, relative to prior to said administering.

In some more particular embodiments, the subject shows a histologic improvement of at least 20%, such as at least 30%, such as at least 40%, such as at least 50%, relative to prior to said administering, wherein:
a) following said administering, the subject shows a decrease in histologic score of at least 10%, such as at least 15%, such as at least 20%, such as at least 30%, such as at least 40%, relative to prior to said administering.
b) following said administering, the subject shows an endoscopic mucosal healing of at least 20%, such as at least 30%, such as at least 40%, relative to prior to said administering;
c) following said administering, the subject shows a decrease in the level of an inflammatory marker relative to the level of the inflammatory marker prior to said administering; and/or
d) following said administering, the subject shows an increase in the level of an inflammatory a marker relative to the level the inflammatory marker prior to said administering;

provided that the inflammatory marker in c) and the inflammatory marker in d) are not the same.

In some embodiments provided herein is a method for treating a condition (or one or more symptoms thereof) characterized by an abnormal inflammatory response in a subject in need thereof are provided (e.g., an autoimmune disorder, e.g., an inflammatory bowel disease) in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows a decrease in histologic score of at least 10%, such as at least 15%, such as at least 20%, such as at least 30%, such as at least 40%, relative to prior to said administering.

In some more particular embodiments, the subject shows a decrease in histologic score of at least 10%, such as at least 15%, such as at least 20%, such as at least 30%, such as at least 40%, relative to prior to said administering, wherein:
a) following said administering, the subject shows an endoscopic mucosal healing of at least 20%, such as at least 30%, such as at least 40%, relative to prior to said administering;
b) following said administering, the subject shows a decrease in the level of an inflammatory marker relative to the level of the inflammatory marker prior to said administering; and/or
c) following said administering, the subject shows an increase in the level of an inflammatory a marker relative to the level the inflammatory marker prior to said administering;

provided that the inflammatory marker in b) and the inflammatory marker in c) are not the same.

In some embodiments provided herein is a method for treating a condition (or one or more symptoms thereof) characterized by an abnormal inflammatory response in a subject in need thereof are provided (e.g., an autoimmune disorder, e.g., an inflammatory bowel disease) in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows an endoscopic mucosal healing of at least 20%, such as at least 30%, such as at least 40%, relative to prior to said administering.

In some more particular embodiments, the subject shows a histologic improvement of at least 20%, such as at least 30%, such as at least 40%, such as at least 50%, relative to prior to said administering, wherein:
e) following said administering, the subject shows a decrease in the level of an inflammatory marker relative to the level of the inflammatory marker prior to said administering; and/or
f) following said administering, the subject shows an increase in the level of an inflammatory a marker relative to the level the inflammatory marker prior to said administering;

provided that the inflammatory marker in a) and the inflammatory marker in b) are not the same.

In some embodiments provided herein is a method for treating a condition (or one or more symptoms thereof) characterized by an abnormal inflammatory response in a subject in need thereof are provided (e.g., an autoimmune disorder, e.g., an inflammatory bowel disease) in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows a decrease in the level of an inflammatory marker relative to the level of the inflammatory marker prior to said administering.

In some more particular embodiments, the level of the inflammatory marker is the level of the marker in colonic mucosa. In some more particular embodiments, the level of the inflammatory marker is the level of the marker in plasma. In some more particular embodiments, the inflammatory marker is TNFα. In some more particular embodiments, the inflammatory marker is IL-12. In some more particular embodiments, the decrease in the level of the inflammatory marker following said administering relative to the level of the inflammatory marker prior to said administering is of at least 5%, such as at least 10%, such as at least 15%, such as at least 20%, such as at least 30%, such as at least 40%, such as at least 50%, such as at least 60%, such as at least 70%, such as at least 80%.

In some embodiments provided herein is a method for treating a condition (or one or more symptoms thereof) characterized by an abnormal inflammatory response in a subject in need thereof are provided (e.g., an autoimmune disorder, e.g., an inflammatory bowel disease) in a subject in need thereof, the method comprising administering by enema an effective amount of a formulation prepared by mixing together a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said administering the subject shows an increase in the level of an inflammatory a marker relative to the level the inflammatory marker prior to said administering.

In some more particular embodiments, the subject that has been treated with an aminosalicylate drug prior to said administering. has not been responsive to the treatment with the aminosalicylate drug.

As used herein an “aminosalicylate drug” is 5-aminosalicylic acid or a compound containing a salicylic acid moiety with a nitrogen substituent at the 5 position. Examples of “aminosalicylate drugs” are:

Balsalazide (Colazal, Giazo)

Mesalamine (Apriso, Asacol, Delzicol, Lialda, Pentasa)

Olsalazine (Dipentum)

Sulfasalazine (Azulfidine, Sulfazine).

In some more particular embodiments wherein the subject has a modified Mayo score (MMS) equal to less than 2 points following said administering, the subject has each subscore of the modified Mayo score (MMS) equal to less than 1 point following said administering,

In some more particular embodiments wherein the subject has a modified Mayo score (MMS) equal to less than 2 points following said administering, the subject has each subscore of the modified Mayo score (MMS) equal to less than 1 point following said administering,

In some more particular embodiments, wherein the subject has at least one of:
(I) a modified Mayo score (MMS) following said administering that is at least 2 points lower than a modified Mayo score (MMS) in the subject prior to said administering;
(II) a modified Mayo score (MMS) following said administering that is at least 25% lower than a modified Mayo score (MMS) in the subject prior to said administering;
(III) a Rectal Bleeding Score (RBS) following said administering that is at least 1 point lower than a Rectal Bleeding Score (RBS) in the subject prior to said administering; or

a Rectal Bleeding Score (RBS) following said administering that is 0 or 1, the subject has at least two of (I), (II), (III) or (IV). In some more particular embodiments, the subject has at least three of (I), (II), (III) or (IV). In some more particular embodiments, the subject has (I), (II), (III) and (IV).

In some more particular embodiments, wherein the subject has at least one of:
(IV) a modified Mayo score (MMS) following said administering that is at least 2 points lower than a modified Mayo score (MMS) in the subject prior to said administering;
(V) a modified Mayo score (MMS) following said administering that is at least 25% lower than a modified Mayo score (MMS) in the subject prior to said administering;
(VI) a Rectal Bleeding Score (RBS) following said administering that is at least 1 point lower than a Rectal Bleeding Score (RBS) in the subject prior to said administering; or

a Rectal Bleeding Score (RBS) following said administering that is 0 or 1, the subject has at least two of (I), (II), (III) or (IV). In some more particular embodiments, the subject has at least three of (I), (II), (III) or (IV). In some more particular embodiments, the subject has (I), (II), (III) and (IV).

In certain embodiments, provided herein is

A) a method for inducing cell death of one or more T cells as disclosed herein (e.g., in the digestive and/or gastrointestinal tract (GI), skin, eyes, or joints), of a subject in need thereof; or

B) a method for treating a subject having a condition associated with unregulated (abnormal, elevated) recruitment and/or retention of one or more T cells as disclosed herein (e.g., at the digestive and/or gastrointestinal tract (GI), skin, eyes, or joints) of a subject;

the method comprising contacting the one or more T cells with an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject is a subject previously diagnosed with ulcerative proctitis and/or proctosigmoiditis.

In some embodiments of the methods herein, the colitis is selected from the group consisting of colitis induced by treatment with adoptive cell therapy, colitis associated by one or more alloimmune diseases (such as graft-vs-host disease, e.g., acute graft vs. host disease and chronic graft vs. host disease), collagenous colitis, lymphocytic colitis, and microscopic colitis.

In certain embodiments, provided herein is

A) a method for inducing cell death of one or more T cells as disclosed herein (e.g., in the digestive and/or gastrointestinal tract (GI), skin, eyes, or joints), of a subject in need thereof; or

B) a method for treating a subject having a condition associated with unregulated (abnormal, elevated) recruitment and/or retention of one or more T cells as disclosed herein (e.g., at the digestive and/or gastrointestinal tract (GI), skin, eyes, or joints) of a subject;

the method comprising contacting the one or more T cells with an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject is a subject previously diagnosed with ulcerative proctitis and/or proctosigmoiditis.

In some embodiments the subject has been diagnosed with ulcerative proctitis and/or proctosigmoiditis at least three months prior to said contacting.

In some more particular embodiments, the subject has been diagnosed with ulcerative proctitis and/or proctosigmoiditis at least three months prior to said contacting, wherein:
g) the subject has a modified Mayo score (MMS) equal to from 4 points or greater than 4 points to 8 points or less than 8 points prior to said contacting;
h) the subject has been treated with an aminosalicylate drug prior to said contacting.
i) the subject has a modified Mayo score (MMS) equal to less than 2 points following said contacting.
j) the subject has at least one of:
k) a modified Mayo score (MMS) following said contacting that is at least 2 points lower than a modified Mayo score (MMS) in the subject prior to said contacting;
l) a modified Mayo score (MMS) following said contacting that is at least 25% lower than a modified Mayo score (MMS) in the subject prior to said contacting;
m) a Rectal Bleeding Score (RBS) following said contacting that is at least 1 point lower than a Rectal Bleeding Score (RBS) in the subject prior to said contacting; or
n) a Rectal Bleeding Score (RBS) following said contacting that is 0 or 1;
o) the subject does not have a modified endoscopic subscore of 0 or 1 prior to said contacting and the subject has a modified endoscopic subscore of 0 or 1 following said contacting;
p) following said contacting, the subject shows a histologic improvement of at least 20%, such as at least 30%, such as at least 40%, such as at least 50%, relative to prior to said contacting;
q) following said contacting, the subject shows a decrease in histologic score of at least 10%, such as at least 15%, such as at least 20%, such as at least 30%, such as at least 40%, relative to prior to said contacting.
r) following said contacting, the subject shows an endoscopic mucosal healing of at least 20%, such as at least 30%, such as at least 40%, relative to prior to said contacting;
s) following said contacting, the subject shows a decrease in the level of an inflammatory marker relative to the level of the inflammatory marker prior to said contacting; and/or
t) following said contacting, the subject shows an increase in the level of an inflammatory a marker relative to the level the inflammatory marker prior to said contacting;
provided that the inflammatory marker in i) and the inflammatory marker in j) are not the same.

In certain embodiments, provided herein is

A) a method for inducing cell death of one or more T cells as disclosed herein (e.g., in the digestive and/or gastrointestinal tract (GI), skin, eyes, or joints), of a subject in need thereof; or

B) a method for treating a subject having a condition associated with unregulated (abnormal, elevated) recruitment and/or retention of one or more T cells as disclosed herein (e.g., at the digestive and/or gastrointestinal tract (GI), skin, eyes, or joints) of a subject;

the method comprising contacting the one or more T cells with an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has a modified Mayo score (MMS) equal to from 4 points or greater than 4 points to 8 points or less than 8 points prior to said contacting.

In some more particular embodiments, the subject has a modified Mayo score (MMS) equal to from 4 points or greater than 4 points to 8 points or less than 8 points prior to said contacting, wherein:
u) the subject has been treated with an aminosalicylate drug prior to said contacting.
v) the subject has a modified Mayo score (MMS) equal to less than 2 points following said contacting.
w) the subject has at least one of:
x) a modified Mayo score (MMS) following said contacting that is at least 2 points lower than a modified Mayo score (MMS) in the subject prior to said contacting;
y) a modified Mayo score (MMS) following said contacting that is at least 25% lower than a modified Mayo score (MMS) in the subject prior to said contacting;
z) a Rectal Bleeding Score (RBS) following said contacting that is at least 1 point lower than a Rectal Bleeding Score (RBS) in the subject prior to said contacting; or
aa) a Rectal Bleeding Score (RBS) following said contacting that is 0 or 1;
bb) the subject does not have a modified endoscopic subscore of 0 or 1 prior to said contacting and the subject has a modified endoscopic subscore of 0 or 1 following said contacting;
cc) following said contacting, the subject shows a histologic improvement of at least 20%, such as at least 30%, such as at least 40%, such as at least 50%, relative to prior to said contacting;
dd) following said contacting, the subject shows a decrease in histologic score of at least 10%, such as at least 15%, such as at least 20%, such as at least 30%, such as at least 40%, relative to prior to said contacting.
ee) following said contacting, the subject shows an endoscopic mucosal healing of at least 20%, such as at least 30%, such as at least 40%, relative to prior to said contacting;
ff) following said contacting, the subject shows a decrease in the level of an inflammatory marker relative to the level of the inflammatory marker prior to said contacting; and/or
gg) following said contacting, the subject shows an increase in the level of an inflammatory a marker relative to the level the inflammatory marker prior to said contacting;
provided that the inflammatory marker in h) and the inflammatory marker in i) are not the same.

In certain embodiments, provided herein is

A) a method for inducing cell death of one or more T cells as disclosed herein (e.g., in the digestive and/or gastrointestinal tract (GI), skin, eyes, or joints), of a subject in need thereof; or

B) a method for treating a subject having a condition associated with unregulated (abnormal, elevated) recruitment and/or retention of one or more T cells as disclosed herein (e.g., at the digestive and/or gastrointestinal tract (GI), skin, eyes, or joints) of a subject;

the method comprising contacting the one or more T cells with an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has been treated with an aminosalicylate drug prior to said contacting.

In some more particular embodiments, the subject has been treated with an aminosalicylate drug prior to said contacting, wherein:
hh) the subject has a modified Mayo score (MMS) equal to less than 2 points following said contacting.
ii) the subject has at least one of:
jj) a modified Mayo score (MMS) following said contacting that is at least 2 points lower than a modified Mayo score (MMS) in the subject prior to said contacting;
kk) a modified Mayo score (MMS) following said contacting that is at least 25% lower than a modified Mayo score (MMS) in the subject prior to said contacting;
ll) a Rectal Bleeding Score (RBS) following said contacting that is at least 1 point lower than a Rectal Bleeding Score (RBS) in the subject prior to said contacting; or
mm) a Rectal Bleeding Score (RBS) following said contacting that is 0 or 1;
nn) the subject does not have a modified endoscopic subscore of 0 or 1 prior to said contacting and the subject has a modified endoscopic subscore of 0 or 1 following said contacting;
oo) following said contacting, the subject shows a histologic improvement of at least 20%, such as at least 30%, such as at least 40%, such as at least 50%, relative to prior to said contacting;
pp) following said contacting, the subject shows a decrease in histologic score of at least 10%, such as at least 15%, such as at least 20%, such as at least 30%, such as at least 40%, relative to prior to said contacting.
qq) following said contacting, the subject shows an endoscopic mucosal healing of at least 20%, such as at least 30%, such as at least 40%, relative to prior to said contacting;
rr) following said contacting, the subject shows a decrease in the level of an inflammatory marker relative to the level of the inflammatory marker prior to said contacting; and/or
ss) following said contacting, the subject shows an increase in the level of an inflammatory a marker relative to the level the inflammatory marker prior to said contacting;

provided that the inflammatory marker in g) and the inflammatory marker in h) are not the same.

In certain embodiments, provided herein is

A) a method for inducing cell death of one or more T cells as disclosed herein (e.g., in the digestive and/or gastrointestinal tract (GI), skin, eyes, or joints), of a subject in need thereof; or

B) a method for treating a subject having a condition associated with unregulated (abnormal, elevated) recruitment and/or retention of one or more T cells as disclosed herein (e.g., at the digestive and/or gastrointestinal tract (GI), skin, eyes, or joints) of a subject;

the method comprising contacting the one or more T cells with an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has a modified Mayo score (MMS) equal to less than 2 points following said contacting.

In some more particular embodiments, the subject has a modified Mayo score (MMS) equal to less than 2 points following said contacting, wherein:
h) the subject has at least one of:
   tt) a modified Mayo score (MMS) following said contacting that is at least 2 points lower than a modified Mayo score (MMS) in the subject prior to said contacting;
   uu) a modified Mayo score (MMS) following said contacting that is at least 25% lower than a modified Mayo score (MMS) in the subject prior to said contacting;
   vv) a Rectal Bleeding Score (RBS) following said contacting that is at least 1 point lower than a Rectal Bleeding Score (RBS) in the subject prior to said contacting; or
   ww) a Rectal Bleeding Score (RBS) following said contacting that is 0 or 1;
i) the subject does not have a modified endoscopic subscore of 0 or 1 prior to said contacting and the subject has a modified endoscopic subscore of 0 or 1 following said contacting;
j) following said contacting, the subject shows a histologic improvement of at least 20%, such as at least 30%, such as at least 40%, such as at least 50%, relative to prior to said contacting;
k) following said contacting, the subject shows a decrease in histologic score of at least 10%, such as at least 15%, such as at least 20%, such as at least 30%, such as at least 40%, relative to prior to said contacting.
l) following said contacting, the subject shows an endoscopic mucosal healing of at least 20%, such as at least 30%, such as at least 40%, relative to prior to said contacting;
m) following said contacting, the subject shows a decrease in the level of an inflammatory marker relative to the level of the inflammatory marker prior to said contacting; and/or
n) following said contacting, the subject shows an increase in the level of an inflammatory a marker relative to the level the inflammatory marker prior to said contacting;

provided that the inflammatory marker in f) and the inflammatory marker in g) are not the same.

In certain embodiments, provided herein is

A) a method for inducing cell death of one or more T cells as disclosed herein (e.g., in the digestive and/or gastrointestinal tract (GI), skin, eyes, or joints), of a subject in need thereof; or

B) a method for treating a subject having a condition associated with unregulated (abnormal, elevated) recruitment and/or retention of one or more T cells as disclosed herein (e.g., at the digestive and/or gastrointestinal tract (GI), skin, eyes, or joints) of a subject;

the method comprising contacting the one or more T cells with an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject has at least one of:
xx) a modified Mayo score (MMS) following said contacting that is at least 2 points lower than a modified Mayo score (MMS) in the subject prior to said contacting;
yy) a modified Mayo score (MMS) following said contacting that is at least 25% lower than a modified Mayo score (MMS) in the subject prior to said contacting;
zz) a Rectal Bleeding Score (RBS) following said contacting that is at least 1 point lower than a Rectal Bleeding Score (RBS) in the subject prior to said contacting; or
aaa) a Rectal Bleeding Score (RBS) following said contacting that is 0 or 1.

In some more particular embodiments, wherein the subject has at least one of (I), (II0, (III) or (IV), wherein:
bbb) the subject does not have a modified endoscopic subscore of 0 or 1 prior to said contacting and the subject has a modified endoscopic subscore of 0 or 1 following said contacting;
ccc) following said contacting, the subject shows a histologic improvement of at least 20%, such as at least 30%, such as at least 40%, such as at least 50%, relative to prior to said contacting;
ddd) following said contacting, the subject shows a decrease in histologic score of at least 10%, such as at least 15%, such as at least 20%, such as at least 30%, such as at least 40%, relative to prior to said contacting.
eee) following said contacting, the subject shows an endoscopic mucosal healing of at least 20%, such as at least 30%, such as at least 40%, relative to prior to said contacting;
fff) following said contacting, the subject shows a decrease in the level of an inflammatory marker relative to the level of the inflammatory marker prior to said contacting; and/or
ggg) following said contacting, the subject shows an increase in the level of an inflammatory a marker relative to the level the inflammatory marker prior to said contacting;

provided that the inflammatory marker in e) and the inflammatory marker in f) are not the same.

In certain embodiments, provided herein is

A) a method for inducing cell death of one or more T cells as disclosed herein (e.g., in the digestive and/or gastrointestinal tract (GI), skin, eyes, or joints), of a subject in need thereof; or

B) a method for treating a subject having a condition associated with unregulated (abnormal, elevated) recruitment and/or retention of one or more T cells as disclosed herein (e.g., at the digestive and/or gastrointestinal tract (GI), skin, eyes, or joints) of a subject;

the method comprising contacting the one or more T cells with an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein the subject does not have a modified endoscopic subscore of 0 or 1 prior to said contacting and the subject has a modified endoscopic subscore of 0 or 1 following said contacting.

In some more particular embodiments, the subject does not have a modified endoscopic subscore of 0 or 1 prior to said contacting and the subject has a modified endoscopic subscore of 0 or 1 following said contacting, wherein:
hhh) following said contacting, the subject shows a histologic improvement of at least 20%, such as at least 30%, such as at least 40%, such as at least 50%, relative to prior to said contacting;
iii) following said contacting, the subject shows a decrease in histologic score of at least 10%, such as at least 15%, such as at least 20%, such as at least 30%, such as at least 40%, relative to prior to said contacting.
jjj) following said contacting, the subject shows an endoscopic mucosal healing of at least 20%, such as at least 30%, such as at least 40%, relative to prior to said contacting;
kkk) following said contacting, the subject shows a decrease in the level of an inflammatory marker relative to the level of the inflammatory marker prior to said contacting; and/or
lll) following said contacting, the subject shows an increase in the level of an inflammatory a marker relative to the level the inflammatory marker prior to said contacting;

provided that the inflammatory marker in d) and the inflammatory marker in e) are not the same.

In certain embodiments, provided herein is

A) a method for inducing cell death of one or more T cells as disclosed herein (e.g., in the digestive and/or gastrointestinal tract (GI), skin, eyes, or joints), of a subject in need thereof; or

B) a method for treating a subject having a condition associated with unregulated (abnormal, elevated) recruitment and/or retention of one or more T cells as disclosed herein (e.g., at the digestive and/or gastrointestinal tract (GI), skin, eyes, or joints) of a subject;

the method comprising contacting the one or more T cells with an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said contacting the subject shows a histologic improvement of at least 20%, such as at least 30%, such as at least 40%, such as at least 50%, relative to prior to said contacting.

In some more particular embodiments, the subject shows a histologic improvement of at least 20%, such as at least 30%, such as at least 40%, such as at least 50%, relative to prior to said contacting, wherein:
mmm) following said contacting, the subject shows a decrease in histologic score of at least 10%, such as at least 15%, such as at least 20%, such as at least 30%, such as at least 40%, relative to prior to said contacting.
nnn) following said contacting, the subject shows an endoscopic mucosal healing of at least 20%, such as at least 30%, such as at least 40%, relative to prior to said contacting;
ooo) following said contacting, the subject shows a decrease in the level of an inflammatory marker relative to the level of the inflammatory marker prior to said contacting; and/or
ppp) following said contacting, the subject shows an increase in the level of an inflammatory a marker relative to the level the inflammatory marker prior to said contacting;

provided that the inflammatory marker in c) and the inflammatory marker in d) are not the same.

In certain embodiments, provided herein is

A) a method for inducing cell death of one or more T cells as disclosed herein (e.g., in the digestive and/or gastrointestinal tract (GI), skin, eyes, or joints), of a subject in need thereof; or

B) a method for treating a subject having a condition associated with unregulated (abnormal, elevated) recruitment and/or retention of one or more T cells as disclosed herein (e.g., at the digestive and/or gastrointestinal tract (GI), skin, eyes, or joints) of a subject;

the method comprising contacting the one or more T cells with an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said contacting the subject shows a decrease in histologic score of at least 10%, such as at least 15%, such as at least 20%, such as at least 30%, such as at least 40%, relative to prior to said contacting.

In some more particular embodiments, the subject shows a decrease in histologic score of at least 10%, such as at least 15%, such as at least 20%, such as at least 30%, such as at least 40%, relative to prior to said contacting, wherein:
qqq) following said contacting, the subject shows an endoscopic mucosal healing of at least 20%, such as at least 30%, such as at least 40%, relative to prior to said contacting;
rrr) following said contacting, the subject shows a decrease in the level of an inflammatory marker relative to the level of the inflammatory marker prior to said contacting; and/or
sss) following said contacting, the subject shows an increase in the level of an inflammatory a marker relative to the level the inflammatory marker prior to said contacting;

provided that the inflammatory marker in b) and the inflammatory marker in c) are not the same.

In certain embodiments, provided herein is

A) a method for inducing cell death of one or more T cells as disclosed herein (e.g., in the digestive and/or gastrointestinal tract (GI), skin, eyes, or joints), of a subject in need thereof; or

B) a method for treating a subject having a condition associated with unregulated (abnormal, elevated) recruitment and/or retention of one or more T cells as disclosed herein (e.g., at the digestive and/or gastrointestinal tract (GI), skin, eyes, or joints) of a subject;

the method comprising contacting the one or more T cells with an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said contacting the subject shows an endoscopic mucosal healing of at least 20%, such as at least 30%, such as at least 40%, relative to prior to said contacting.

In some more particular embodiments, the subject shows a histologic improvement of at least 20%, such as at least 30%, such as at least 40%, such as at least 50%, relative to prior to said contacting, wherein:
ttt) following said contacting, the subject shows a decrease in the level of an inflammatory marker relative to the level of the inflammatory marker prior to said contacting; and/or
uuu) following said contacting, the subject shows an increase in the level of an inflammatory a marker relative to the level the inflammatory marker prior to said contacting;

provided that the inflammatory marker in a) and the inflammatory marker in b) are not the same.

In certain embodiments, provided herein is

A) a method for inducing cell death of one or more T cells as disclosed herein (e.g., in the digestive and/or gastrointestinal tract (GI), skin, eyes, or joints), of a subject in need thereof; or

B) a method for treating a subject having a condition associated with unregulated (abnormal, elevated) recruitment and/or retention of one or more T cells as disclosed herein (e.g., at the digestive and/or gastrointestinal tract (GI), skin, eyes, or joints) of a subject;

the method comprising contacting the one or more T cells with an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof; one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said contacting the subject shows a decrease in the level of an inflammatory marker relative to the level of the inflammatory marker prior to said contacting.

In some more particular embodiments, the level of the inflammatory marker is the level of the marker in colonic mucosa. In some more particular embodiments, the level of the inflammatory marker is the level of the marker in plasma. In some more particular embodiments, the inflammatory marker is TNFα. In some more particular embodiments, the inflammatory marker is IL-12. In some more particular embodiments, the decrease in the level of the inflammatory marker following said contacting relative to the level of the inflammatory marker prior to said contacting is of at least 5%, such as at least 10%, such as at least 15%, such as at least 20%, such as at least 30%, such as at least 40%, such as at least 50%, such as at least 60%, such as at least 70%, such as at least 80%.

In certain embodiments, provided herein is

A) a method for inducing cell death of one or more T cells as disclosed herein (e.g., in the digestive and/or gastrointestinal tract (GI), skin, eyes, or joints), of a subject in need thereof; or

B) a method for treating a subject having a condition associated with unregulated (abnormal, elevated) recruitment and/or retention of one or more T cells as disclosed herein (e.g., at the digestive and/or gastrointestinal tract (GI), skin, eyes, or joints) of a subject;

the method comprising contacting the one or more T cells with an effective amount of a formulation comprising a first component and a second component, wherein:

(i) the first component comprises a solid pharmaceutical composition, which comprises:

an inner phase which is a wet granulated solid preparation comprising niclosamide, or a pharmaceutically acceptable salt thereof, one or more disintegrants; one or more diluents; and one or more binders;

an external phase comprising one or more glidants and/or one or more lubricants; and

(ii) the second component comprises one or more liquids and optionally one or more other pharmaceutically acceptable excipients together forming a liquid carrier,

wherein following said contacting the subject shows an increase in the level of an inflammatory a marker relative to the level the inflammatory marker prior to said contacting.

In some particular embodiments, the colitis is autoimmune colitis (or one or more symptoms thereof).

In some embodiments of the methods herein, the colitis is selected from the group consisting of colitis induced by treatment with adoptive cell therapy, colitis associated by one or more alloimmune diseases (such as graft-vs-host disease, e.g., acute graft vs. host disease and chronic graft vs. host disease), collagenous colitis, lymphocytic colitis, and microscopic colitis.

In some more particular embodiments, the subject that has been treated with an aminosalicylate drug prior to said contacting. has not been responsive to the treatment with the aminosalicylate drug.

As used herein an “aminosalicylate drug” is 5-aminosalicylic acid or a compound containing a salicylic acid moiety with a nitrogen substituent at the 5 position. Examples of “aminosalicylate drugs” are:

Balsalazide (Colazal, Giazo)

Mesalamine (Apriso, Asacol, Delzicol, Lialda, Pentasa)

Olsalazine (Dipentum)

Sulfasalazine (Azulfidine, Sulfazine).

In some more particular embodiments wherein the subject has a modified Mayo score (MMS) equal to less than 2 points following said contacting, the subject has each subscore of the modified Mayo score (MMS) equal to less than 1 point following said contacting,

In some more particular embodiments, wherein the subject has at least one of:
(VII) a modified Mayo score (MMS) following said contacting that is at least 2 points lower than a modified Mayo score (MMS) in the subject prior to said contacting;
(VIII) a modified Mayo score (MMS) following said contacting that is at least 25% lower than a modified Mayo score (MMS) in the subject prior to said contacting;
(IX) a Rectal Bleeding Score (RBS) following said contacting that is at least 1 point lower than a Rectal Bleeding Score (RBS) in the subject prior to said contacting; or

a Rectal Bleeding Score (RBS) following said contacting that is 0 or 1, the subject has at least two of (I), (II), (III) or (IV). In some more particular embodiments, the subject has at least three of (I), (II), (III) or (IV). In some more particular embodiments, the subject has (I), (II), (III) and (IV).

In some more particular embodiments, wherein the subject has at least one of:
(X) a modified Mayo score (MMS) following said contacting that is at least 2 points lower than a modified Mayo score (MMS) in the subject prior to said contacting;
(XI) a modified Mayo score (MMS) following said contacting that is at least 25% lower than a modified Mayo score (MMS) in the subject prior to said contacting;
(XII) a Rectal Bleeding Score (RBS) following said contacting that is at least 1 point lower than a Rectal Bleeding Score (RBS) in the subject prior to said contacting; or

a Rectal Bleeding Score (RBS) following said contacting that is 0 or 1, the subject has at least two of (I), (II), (III) or (IV). In some more particular embodiments, the subject has at least three of (I), (II), (III) or (IV). In some more particular embodiments, the subject has (I), (II), (III) and (IV).

In some embodiments of the methods herein, the formulation is administered by enema.

In some embodiments of the methods herein, the formulation is prepared by mixing together the first component and the second component.

In some embodiments of the methods herein, niclosamide or a pharmaceutically acceptable salt thereof is administered in an amount of 150 mg twice daily.

In some embodiments of the methods herein, niclosamide or a pharmaceutically acceptable salt thereof is administered in an amount of 450 mg twice daily.

In some embodiments of the methods herein, niclosamide or a pharmaceutically acceptable salt thereof is administered in an amount of 900 mg once daily.

This disclosure contemplates both monotherapy regimens as well as combination therapy regimens.

In some embodiments, inducing cell death of the one or more T cells includes one or more of the following pathways: Programmed cell death, Necroptosis, Apoptosis, Necrosis, Pyroptosis, Ferroptosis, Anoikis, Mitotic catastrophe, Paraptosis, Pyronecrosis, Entosis, Netosis, Parthanatos, Autophagic cell death, RGD: regulated cell death, Non-apoptotic programmed cell-death, Caspase-independent programmed cell-death inducing necrosis or apoptosis of the one or more T cells, e.g., necrosis or apoptosis of the one or more T cells. In certain embodiments, the effective amount is an amount sufficient to induce cell death of at least one of the one or more T cells (e.g., by any one or more of the pathways described above, e.g., necrosis or apoptosis of the one or more T cells).

In some embodiments, the one or more T cells include one or more activated T cells, e.g., one or more activated T cells is independently selected from the group consisting of:
CD45+CD3+TCRαβ+CD62L−;
CD45+CD3+TCRαβ+CD62L-CCR7−;
CD45+CD3+TCRαβ+CD62L-CD69+;
CD45+CD3+TCRαβ+CD62L-CD69+PD-1+;
CD45+CD3+TCRαβ+CD62L-CTLA4+;
CD45+CD3+TCRαβ+CD62L-PD-1++CTLA4+;
CD45+CD3+TCRγδ+CD62L−;
CD45+CD3+TCRγδ+CD62L-CCR7−;
CD45+CD3+TCRγδ+CD62L-CD69+;
CD45+CD3+TCRγδ+CD62L-CD69+PD-1+;
CD45+CD3+CD62L−TCRγδ+CTLA4+; and
CD45+CD3+TCRγδ+CD62L-PD-1++CTLA4+.

In certain embodiments, the effective amount is an amount sufficient to induce cell death of at least one of the one or more activated T cells (e.g., by any one or more of the pathways described above, e.g., necrosis or apoptosis of the one or more activated T cells).

In some embodiments, the one or more T cells are present within the intestinal epithelium and/or within the Lamina propria and/or within the Peyer's patches (PP) and/or within the GALT (gut associated lymphoid tissue) and/or within the intestinal mucosa and/or within the intestinal submucosa and/or within the intestinal muscular layer and/or within the intestinal serosa.

In some embodiments, the one or more T cells comprise one or more gut tropic T cells. In certain embodiments, each of the one or more gut tropic T cells independently expresses one or more gut-homing receptors selected from the group consisting of:
(CD3+CCR9+;
CD3+α4+ or CD3+β7+;
CD3+α4+β7+;
CD3+β1+;
CD3+α4+β1+;
CD3+LFA1;
CD3+CCR4+; and
CD3+CCR10+.

In certain of these embodiments, the condition is an autoimmune disease.

Non-limiting examples of autoimmune diseases include: arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis (e.g., Hashimoto's thyroiditis), dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis.

In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In certain embodiments, the condition is autoimmune colitis.

In certain of these embodiments, the autoimmune colitis is induced by one or more chemotherapeutic agents, e.g., a chemotherapeutic immunomodulator, e.g., an immune checkpoint inhibitor. In certain of these embodiments, the immune checkpoint inhibitor targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine—TIM3, lymphocyte activation gene 3 protein (LAG3), MHC class II—LAG3, 4-1BB-4-1BB ligand, OX40-OX40 ligand, GITR, GITR ligand—GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine—TIM3, SIRPA-CD47, VEGF, Neuropilin, CD160, CD30, and CD155; e.g., CTLA-4 or PD1 or PD-L1). See, e.g., Postow, M. J. Clin. Oncol. 2015, 33, 1.

In certain of these embodiments, the immune checkpoint inhibitor is selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A, and MGA271.

In certain of these embodiments, the immune checkpoint inhibitor targets CTLA-4, e.g., an antibody, e.g., ipilimumab or tremelimumab.

In certain of these embodiments, the immune checkpoint inhibitor targets PD1 or PD-L1, e.g., nivolumab, lambrolizumab, or BMS-936559.

In certain embodiments, the condition is mucositis, also known as stomatitis, which can occur as a result of chemotherapy or radiation therapy, either alone or in combination as well as damage caused by exposure to radiation outside of the context of radiation therapy. Chemotherapeutic agents which may induce mucositis when used alone or in combination include, but are not limited to, platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

In certain embodiments, the condition is uveitis, which is inflammation of the uvea (e.g., anterior uveitis, e.g., iridocyclitis or iritis; intermediate uveitis (also known as pars planitis); posterior uveitis; or chorioretinitis, e.g., pan-uveitis).

In some embodiments, monotherapy includes administering (e.g., topically and locally) to a subject an effective amount of a chemical entity (e.g., a compound exhibiting activity as a mitochondrial uncoupling agent or a pharmaceutically acceptable salt and/or hydrate and/or cocrystal thereof; e.g., a compound, such as niclosamide or a pharmaceutically acceptable salt and/or hydrate and/or cocrystal thereof, e.g., a compound, such as a niclosamide analog, or a pharmaceutically acceptable salt and/or hydrate and/or cocrystal thereof) as defined anywhere herein, but excludes the administration of other therapeutic agents (e.g., the active compounds, e.g., peptides, disclosed in U.S. Pat. No. 8,148,328, which is incorporated herein by reference in its entirety).

In some embodiments, the methods described herein can further include administering a second therapeutic agent or regimen.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

In certain embodiments, the second therapeutic agent is a chemotherapeutic immunomodulator, e.g., an immune checkpoint inhibitor, which can be as defined anywhere herein. In other embodiments, the second therapeutic agent or regimen is one or more anti-inflammatory agents or immunomodulator acting locally in the GI tract. In other embodiments, the second therapeutic agent or regimen is 5-ASA (and associated delivery systems), anti-SMAD7 antisense, orally formulated anti-TNFs, anti-integrins, sulfasalazine, balsalazide, steroids, azathioprine, and methotrexate. In further embodiments, the second therapeutic agent or regimen is radiation or surgery.

In certain embodiments, the second therapeutic agent is platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

In still other embodiments, the second therapeutic agent can be selected from those delineated above (see U.S. Pat. No. 7,927,613, which is incorporated herein by reference in its entirety).

In some embodiments, the methods described herein further include the step of identifying a subject (e.g., a patient) in need of such treatment (e.g., by way of biopsy, endoscopy, or other conventional method known in the art).

In some embodiments, the chemical entities, methods, and compositions described herein can be administered to certain treatment-resistant patient populations, e.g., one that is nonresponsive or resistant to treatment with an anti-TNFalpha therapy (e.g., Humira, Enbrel, Remicade, Cimzia, Simponi, Enbrel, xanthine derivatives, e.g., pentoxifylline and Bupropion; (R)-DOI, TCB-2, LSD and LA-SS-Az). In certain embodiments, the patient is undergoing and/or has undergone treatment with an anti-TNFalpha therapy (e.g., Humira, Enbrel, Remicade, Cimzia, Simponi, Enbrel, xanthine derivatives, e.g., pentoxifylline and Bupropion; (R)-DOI, TCB-2, LSD and LA-SS-Az).

Combination Therapy

In some embodiments, the methods and compositions described herein are suitable for use in combination therapy with various other therapeutic regimens (e.g., chemotherapy and/or radiation). In certain embodiments, the chemical entities and methods described herein can be used to treat side effects produced by such therapeutic regimens, e.g., inflammatory bowel diseases induced by chemotherapeutic immunomodulators, e.g., checkpoint inhibitors, which in some cases can be prohibitively severe.

In some embodiments, the methods and compositions described herein are suitable for use in combination therapy with one or more additional therapeutic agents.

In certain embodiments, the one or more additional therapeutic agents is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the one or more additional therapeutic agents is administered to the subject at about the same time as contacting with or administering the chemical entity. By way of example, the additional therapeutic agent or regimen and the chemical entity are provided to the subject simultaneously in the same dosage form. As another example, the additional therapeutic agent or regimen and the chemical entity are provided to the subject concurrently in separate dosage forms.

In still other embodiments, the one or more additional therapeutic agents is administered to the subject after contacting with or administering the chemical entity (e.g., about one hour after, or about 6 hours after, or about 12 hours after, or about 24 hours after, or about 48 hours after, or about 1 week after, or about 1 month after).

As another example, the one or more therapeutic agents can be: budesonide; epidermal growth factor; corticosteroids; cyclosporine; sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1 monoclonal antibodies; anti-IL-6 monoclonal antibodies (e.g., anti-IL-6 receptor antibodies and anti-IL-6 antibodies); growth factors; elastase inhibitors; pyridinyl-imidazole compounds; TNF antagonists as described herein; IL-4, IL-10, IL-13 and/or TGF.beta. cytokines or agonists thereof (e.g., agonist antibodies); IL-11; glucuronide- or dextran-conjugated prodrugs of prednisolone, dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of platelet activating factor (PAF); ciprofloxacin; and/or lignocaine.

In some embodiments, the methods and compositions described herein are suitable for use in combination therapy with one or more additional therapeutic agents for treating or preventing inflammatory bowel disease (IBS) (e.g., Crohn's disease, ulcerative colitis). Non-limiting examples of the additional therapeutic agents include: sphingosine 1-phosphate (S1P) receptor modulators (e.g., etrasimod or ozanimod); steroidal anti-inflammatory agents (e.g, beclomethasone 17 or budesonide); non-steroidal anti-inflammatory agents (e.g., 5-ASA); receptor-interacting protein kinase 1 (RIPK1) inhibitors (e.g., GSK2982772); EP4 modulators (e.g., KAG-308); toll-like receptor (e.g., TLR4, TLR9) modulators (e.g., JKB-122, cobitolimod); Janus kinase (JAK) inhibitors (e.g., TD-1473, tofacitinib, upadacitinib, filgotinib, PF-06651600, and PF-06700841); lanthionine synthetase C-like 2 (LANCL2) modulators (e.g., BT-11); phosphatidylcholine (e.g., LT-02); integrin (e.g., α4 Integrin) modulators (e.g, AJM-300 (carotegrast)); Smad7 modulators (e.g., mongersen); phosphodiesterase 4 (PDE4) modulators (e.g., apremilast); tumor progression locus 2 (TPL2) inhibitors (e.g., GS-4875); tyrosine kinase 2 (TYK2) inhibitors (e.g., BMS-986165, PF-06700841, and PF-06826647); and TEC kinase inhibitors (e.g., PF-06651600).

In some embodiments, the one or more additional therapeutic agents is selected from an agent/regimen for treating rheumatoid arthritis. Non-limiting examples include non-steroidal anti-inflammatory drugs (NSAIDs; e.g., ibuprofen and naproxen), corticosteroids (e.g, prednisone), disease-modifying antirheumatic drugs (DMARDs; e.g., methotrexate (Trexall®, Otrexup®, Rasuvo®, Rheumatrex®), leflunomide (Arava®), hydroxychloroquine (Plaquenil), PF-06650833, iguratimod, tofacitinib (Xeljanz®), ABBV-599, evobrutinib, and sulfasalazine (Azulfidine®)), and biologics (e.g., abatacept (Orencia®), adalimumab (Humira®), anakinra (Kineret®), certolizumab (Cimzia®), etanercept (Enbrel®), golimumab (Simponi®), infliximab (Remicade®), rituximab (Rituxan®), tocilizumab (Actemra®), vobarilizumab, sarilumab (Kevzara®), secukinumab, ABP 501, CHS-0214, ABC-3373, and tocilizumab (ACTEMRA®)).

In some embodiments, the one or more additional therapeutic agents is selected from an agent/regimen for treating lupus. Non-limiting examples include steroids, topical immunomodulators (e.g., tacrolimus ointment (Protopic®) and pimecrolimus cream (Elidel®)), thalidomide (Thalomid®), non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., evobrutinib, iberdomide, voclosporin, cenerimod, azathioprine (Imuran®), cyclophosphamide (Cytoxan®, Neosar®, Endoxan®), and cyclosporine (Neoral, Sandimmune®, Gengraf®), and mycophenolate mofetil) baricitinib, iguratimod, filgotinib, GS-9876, rapamycin, and PF-06650833), and biologics (e.g., belimumab (Benlysta®), anifrolumab, prezalumab, MEDI0700, obinutuzumab, vobarilizumab, lulizumab, atacicept, PF-06823859, and lupizor, rituximab, BT063, BI655064, BIIB059, aldesleukin (Proleukin®), dapirolizumab, edratide, IFN-α-kinoid, OMS721, RC18, RSLV-132, theralizumab, XmAb5871, and ustekinumab (Stelara®)). For example, non-limiting treatments for systemic lupus erythematosus include non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., iberdomide, voclosporin, azathioprine (Imuran®), cyclophosphamide (Cytoxan®, Neosar®, Endoxan®), and cyclosporine (Neoral, Sandimmune®, Gengraf®), and mycophenolate mofetil, baricitinib, filgotinib, and PF-06650833), and biologics (e.g., belimumab (Benlysta®), anifrolumab, prezalumab, MEDI0700, vobarilizumab, lulizumab, atacicept, PF-06823859, lupizor, rituximab, BT063, BI655064, BIIB059, aldesleukin (Proleukin®), dapirolizumab, edratide, IFN-α-kinoid, RC18, RSLV-132, theralizumab, XmAb5871, and ustekinumab (Stelara®)). As another example, non-limiting examples of treatments for cutaneous lupus include steroids, immunomodulators (e.g., tacrolimus ointment (Protopic®) and pimecrolimus cream (Elidel®)), GS-9876, filgotinib, and thalidomide (Thalomid®). Agents and regimens for treating drug-induced and/or neonatal lupus can also be administered.

In some embodiments, the one or more additional therapeutic agents is selected from an agent/regimen for treating IBDs. Non-limiting examples include 6-mercaptopurine, AbGn-168H, ABX464, ABT-494, adalimumab, AJM300, alicaforsen, AMG139, anrukinzumab, apremilast, ATR-107 (PF0530900), autologous CD34-selected peripheral blood stem cells transplant, azathioprine, bertilimumab, BI 655066, BMS-936557, certolizumab pegol (Cimzia®), cobitolimod, corticosteroids (e.g., prednisone, Methylprednisolone, prednisone), CP-690,550, CT-P13, cyclosporine, DIMS0150, E6007, E6011, etrasimod, etrolizumab, fecal microbial transplantation, filgotinib, fingolimod, firategrast (SB-683699) (formerly T-0047), GED0301, GLPG0634, GLPG0974, guselkumab, golimumab, GSK1399686, HMPL-004 (Andrographis paniculata extract), IMU-838, infliximab, Interleukin 2 (IL-2), Janus kinase (JAK) inhibitors, laquinimod, masitinib (AB1010), matrix metalloproteinase 9 (MMP 9) inhibitors (e.g., GS-5745), MEDI2070, mesalamine, methotrexate, mirikizumab (LY3074828), natalizumab, NNC 0142-0000-0002, NNC0114-0006, ozanimod, peficitinib (JNJ-54781532), PF-00547659, PF-04236921, PF-06687234, QAX576, RHB-104, rifaximin, risankizumab, RPC1063, SB012, SHP647, sulfasalazine, TD-1473, thalidomide, tildrakizumab (MK 3222), TJ301, TNF-Kinoid®, tofacitinib, tralokinumab, TRK-170, upadacitinib, ustekinumab, UTTR1147A, V565, vatelizumab, VB-201, vedolizumab, and vidofludimus.

In some embodiments, the one or more additional therapeutic agents is selected from an agent/regimen for treating irritable bowel syndrome. Non-limiting examples include alosetron, bile acid sequestrants (e.g., cholestyramine, colestipol, colesevelam), chloride channel activators (e.g., lubiprostone), coated peppermint oil capsules, desipramine, dicyclomine, ebastine, eluxadoline, farnesoid X receptor agonist (e.g., obeticholic acid), fecal microbiota transplantation, fluoxetine, gabapentin, guanylate cyclase-C agonists (e.g., linaclotide, plecanatide), ibodutant, imipramine, JCM-16021, loperamide, lubiprostone, nortriptyline, ondansetron, opioids, paroxetine, pinaverium, polyethylene glycol, pregabalin, probiotics, ramosetron, rifaximin, and tanpanor.

In some embodiments, the one or more additional therapeutic agents is selected from an agent/regimen for treating scleroderma. Non-limiting examples include non-steroidal anti-inflammatory drugs (NSAIDs; e.g., ibuprofen and naproxen), corticosteroids (e.g, prednisone), immunomodulators (e.g., azathioprine, methotrexate (Trexall®, Otrexup®, Rasuvo®, Rheumatrex®), cyclophosphamide (Cytoxan®, Neosar®, Endoxan®), and cyclosporine (Neoral®, Sandimmune®, Gengraf®), antithymocyte globulin, mycophenolate mofetil, intravenous immunoglobulin, rituximab, sirolimus, and alefacept), calcium channel blockers (e.g., nifedipine), alpha blockers, serotonin receptor antagonists, angiotensin II receptor inhibitors, statins, local nitrates, iloprost, phosphodiesterase 5 inhibitors (e.g., sildenafil), bosentan, tetracycline antibiotics, endothelin receptor antagonists, prostanoids, and tyrosine kinase inhibitors (e.g., imatinib, nilotinib and dasatinib).

In some embodiments, the one or more additional therapeutic agents is selected from an agent/regimen for treating Crohn's Disease (CD). Non-limiting examples include adalimumab, autologous CD34-selected peripheral blood stem cells transplant, 6-mercaptopurine, azathioprine, certolizumab pegol (Cimzia®), corticosteroids (e.g., prednisone), etrolizumab, E6011, fecal microbial transplantation, filgotinib, guselkumab, infliximab, IL-2, JAK inhibitors, matrix metalloproteinase 9 (MMP 9) inhibitors (e.g., GS-5745), MEDI2070, mesalamine, methotrexate, natalizumab, ozanimod, RHB-104, rifaximin, risankizumab, SHP647, sulfasalazine, thalidomide, upadacitinib, V565, and vedolizumab.

In some embodiments, the one or more additional therapeutic agents is selected from an agent/regimen for treating UC. Non-limiting examples include AbGn-168H, ABT-494, ABX464, apremilast, PF-00547659, PF-06687234, 6-mercaptopurine, adalimumab, azathioprine, bertilimumab, brazikumab (MEDI2070), cobitolimod, certolizumab pegol (Cimzia®), CP-690,550, corticosteroids (e.g., multimax budesonide, Methylprednisolone), cyclosporine, E6007, etrasimod, etrolizumab, fecal microbial transplantation, filgotinib, guselkumab, golimumab, IL-2, IMU-838, infliximab, matrix metalloproteinase 9 (MMP9) inhibitors (e.g., GS-5745), mesalamine, mesalamine, mirikizumab (LY3074828), RPC1063, risankizumab (BI 6555066), SHP647, sulfasalazine, TD-1473, TJ301, tildrakizumab (MK 3222), tofacitinib, tofacitinib, ustekinumab, UTTR1147A, and vedolizumab.

In some embodiments, the one or more additional therapeutic agents is selected from an agent/regimen for treating autoimmune colitis. Non-limiting examples include corticosteroids (e.g., budesonide, prednisone, prednisolone, Beclometasone dipropionate), diphenoxylate/atropine, infliximab, loperamide, mesalamine, TIP60 inhibitors (see, e.g., U.S. Patent Application Publication No. 2012/0202848), and vedolizumab.

In some embodiments, the one or more additional therapeutic agents is selected from an agent/regimen for treating iatrogenic autoimmune colitis. Non-limiting examples include corticosteroids (e.g., budesonide, prednisone, prednisolone, Beclometasone dipropionate), diphenoxylate/atropine, infliximab, loperamide, TIP60 inhibitors (see, e.g., U.S. Patent Application Publication No. 2012/0202848), and vedolizumab.

In some embodiments, the one or more additional therapeutic agents is selected from an agent/regimen for treating colitis induced by one or more chemotherapeutics agents. Non-limiting examples include corticosteroids (e.g., budesonide, prednisone, prednisolone, beclometasone dipropionate), diphenoxylate/atropine, infliximab, loperamide, mesalamine, TIP60 inhibitors (see, e.g., U.S. Patent Application Publication No. 2012/0202848), and vedolizumab.

In some embodiments, the one or more additional therapeutic agents is selected from an agent/regimen for treating colitis induced by treatment with adoptive cell therapy. Non-limiting examples include corticosteroids (e.g., budesonide, prednisone, prednisolone, beclometasone dipropionate), diphenoxylate/atropine, infliximab, loperamide, TIP60 inhibitors (see, e.g., U.S. Patent Application Publication No. 2012/0202848), and vedolizumab.

In some embodiments, the one or more additional therapeutic agents is selected from an agent/regimen for treating colitis associated with one or more alloimmune diseases. Non-limiting examples include corticosteroids (e.g., budesonide, prednisone, prednisolone, beclometasone dipropionate), sulfasalazine, and eicopentaenoic acid.

In some embodiments, the one or more additional therapeutic agents is selected from an agent/regimen for treating radiation enteritis. Non-limiting examples include teduglutide, amifostine, angiotensin-converting enzyme (ACE) inhibitors (e.g., benazepril, captopril, enalapril, fosinopril, lisinopril, moexipril, perindopril, quinapril, ramipril, and trandolapril), probiotics, selenium supplementation, statins (e.g., atorvastatin, fluvastatin, lovastatin, pravastatin, rosuvastatin, simvastatin, and pitavastatin), sucralfate, and vitamin E.

In some embodiments, the one or more additional therapeutic agents is selected from an agent/regimen for treating collagenous colitis. Non-limiting examples include 6-mercaptopurine, azathioprine, bismuth subsalicate, Boswellia serrata extract, cholestyramine, colestipol, corticosteroids (e.g., budesonide, prednisone, prednisolone, beclometasone dipropionate), loperamide, mesalamine, methotrexate, probiotics, and sulfasalazine.

In some embodiments, the one or more additional therapeutic agents is selected from an agent/regimen for treating lyphocytic colitis. Non-limiting examples include 6-mercaptopurine, azathioprine, bismuth subsalicylate, cholestyramine, colestipol, corticosteroids (e.g., budesonide, prednisone, prednisolone, beclometasone dipropionate), loperamide, mesalamine, methotrexate, and sulfasalazine.

In some embodiments, the one or more additional therapeutic agents is selected from an agent/regimen for treating microscopic colitis. Non-limiting examples include 6-mercaptopurine, azathioprine, bismuth subsalicylate, Boswellia serrata extract, cholestyramine, colestipol, corticosteroids (e.g., budesonide, prednisone, prednisolone, beclometasone dipropionate), fecal microbial transplantation, loperamide, mesalamine, methotrexate, probiotics, and sulfasalazine.

In some embodiments, the one or more additional therapeutic agents is selected from an agent/regimen for treating alloimmune disease. Non-limiting examples include intrauterine platelet transfusions, intravenous immunoglobin, maternal steroids, abatacept, alemtuzumab, alpha1-antitrypsin, AMG592, antithymocyte globulin, baricitinib, basiliximab, bortezomib, brentuximab, cannabidiol, corticosteroids (e.g., methylprednisone, prednisone), cyclosporine, daclizumab, defibrotide, denileukin diftitox, glasdegib, ibrutinib, IL-2, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

In some embodiments, the one or more additional therapeutic agents is selected from an agent/regimen for treating multiple sclerosis (MS). Non-limiting examples include alemtuzumab (Lemtrada®), ALKS 8700, amiloride, ATX-MS-1467, azathioprine, baclofen (Lioresal®), beta interferons (e.g., IFN-β-1a, IFN-β-1b), cladribine, corticosteroids (e.g., methylprednisolone), daclizumab, dimethyl fumarate (Tecfidera®), fingolimod (Gilenya®), fluoxetine, glatiramer acetate (Copaxone®), hydroxychloroquine, ibudilast, idebenone, laquinimod, lipoic acid, losartan, masitinib, MD1003 (biotin), mitoxantrone, montelukast, natalizumab (Tysabri®), NeuroVax™, ocrelizumab, ofatumumab, pioglitazone, and RPC1063.

In some embodiments, the one or more additional therapeutic agents is selected from an agent/regimen for treating graft-vs-host disease. Non-limiting examples include abatacept, alemtuzumab, alpha1-antitrypsin, AMG592, antithymocyte globulin, baricitinib, basiliximab, bortezomib, brentuximab, cannabidiol, corticosteroids (e.g., methylprednisone, prednisone), cyclosporine, daclizumab, defibrotide, denileukin diftitox, glasdegib, ibrutinib, IL-2, imatinib, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

In some embodiments, the one or more additional therapeutic agents is selected from an agent/regimen for treating acute graft-vs-host disease. Non-limiting examples include alemtuzumab, alpha-1 antitrypsin, antithymocyte globulin, basiliximab, brentuximab, corticosteroids (e.g., methylprednisone, prednisone), cyclosporine, daclizumab, defibrotide, denileukin diftitox, ibrutinib, infliximab, itacitinib, LBH589, mycophenolate mofetil, natalizumab, neihulizumab, pentostatin, photopheresis, ruxolitinib, sirolimus, tacrolimus, and tocilizumab.

In some embodiments, the one or more additional therapeutic agents is selected from an agent/regimen for treating chronic graft vs. host disease. Non-limiting examples include abatacept, alemtuzumab, AMG592, antithymocyte globulin, basiliximab, bortezomib, corticosteroids (e.g., methylprednisone, prednisone), cyclosporine, daclizumab, denileukin diftitox, glasdegib, ibrutinib, IL-2, imatinib, infliximab, mycophenolate mofetil, pentostatin, photobiomodulation, photopheresis, ruxolitinib, sirolimus, sonidegib, tacrolimus, tocilizumab, and vismodegib.

In some embodiments, the one or more additional therapeutic agents is selected from an agent/regimen for treating celiac disease. Non-limiting examples include AMG 714, AMY01, Aspergillus niger prolyl endoprotease, BL-7010, CALY-002, GBR 830, Hu-Mik-Beta-1, IMGX003, KumaMax, Larazotide Acetate, Nexvan2®, pancrelipase, TIMP-GLIA, vedolizumab, and ZED1227.

In some embodiments, the one or more additional therapeutic agents is selected from an agent/regimen for treating psoriasis. Non-limiting examples include topical corticosteroids, topical crisaborole/AN2728, topical SNA-120, topical SAN021, topical tapinarof, topical tocafinib, topical IDP-118, topical M518101, topical calcipotriene and betamethasone dipropionate (e.g., MC2-01 cream and Taclonex®), topical P-3073, topical LEO 90100 (Enstilar®), topical betamethasone dipropriate (Sernivo®), halobetasol propionate (Ultravate®), vitamin D analogues (e.g., calcipotriene (Dovonex®) and calcitriol (Vectical®)), anthralin (e.g., Dritho-Scalp® and Dritho-Creme®), topical retinoids (e.g., tazarotene (e.g., Tazorac® and Avage®)), calcineurin inhibitors (e.g., tacrolimus (Prograf®) and pimecrolimus (Elidel®)), salicylic acid, coal tar, moisturizers, phototherapy (e.g., exposure to sunlight, UVB phototherapy, narrow band UVB phototherapy, Goeckerman therapy, psoralen plus ultraviolet A (PUVA) therapy, and excimer laser), retinoids (e.g., acitretin (Soriatane®)), methotrexate (Trexall®, Otrexup®, Rasuvo®, Rheumatrex®), Apo805K1, baricitinib, FP187, KD025, prurisol, VTP-43742, XP23829, ZPL-389, CF101 (piclidenoson), LAS41008, VPD-737 (serlopitant), upadacitinib (ABT-494), apremilast, tofacitinib, cyclosporine (Neoral®, Sandimmune®, Gengraf®), biologics (e.g., etanercept (Enbrel®), entanercept-szzs (Elrezi®), infliximab (Remicade®), adalimumab (Humira®), adalimumab-adbm (Cyltezo®), ustekinumab (Stelara®), golimumab (Simponi®), apremilast (Otezla®), secukinumab (Cosentyx®), certolixumab pegol, secukinumab, tildrakizumab-asmn, infliximab-dyyb, abatacept, ixekizumab (Taltz®), ABP 710, BCD-057, BI695501, bimekizumab (UCB4940), CHS-1420, GP2017, guselkumab (CNTO 1959), HD203, M923, MSB 11022, Mirikizumab (LY3074828), PF-06410293, PF-06438179, risankizumab (BI655066), SB2, SB4, SB5, siliq (brodalumab), namilumab (MT203, tildrakizumab (MK-3222), and ixekizumab (Taltz®)), thioguanine, and hydroxyurea (e.g., Droxia® and Hydrea®).

In some embodiments, the one or more additional therapeutic agents is selected from an agent/regimen for treating cutaneous T-cell lymphoma. Non-limiting examples include phototherapy (e.g., exposure to sunlight, UVB phototherapy, narrow band UVB phototherapy, Goeckerman therapy, psoralen plus ultraviolet A (PUVA) therapy, and excimer laser), extracorporeal photopheresis, radiation therapy (e.g., spot radiation and total skin body electron beam therapy), stem cell transplant, corticosteroids, imiquimod, bexarotene gel, topical bis-chloroethyl-nitrourea, mechlorethamine gel, vorinostat (Zolinza®), romidepsin (Istodax®), pralatrexate (Folotyn®) biologics (e.g., alemtuzumab (Campath®), brentuximab vedotin (SGN-35), mogamulizumab, and IPH4102).

In some embodiments, the one or more additional therapeutic agents is selected from an agent/regimen for treating uveitis. Non-limiting examples include corticosteroids (e.g., intravitreal triamcinolone acetonide injectable suspensions), antibiotics, antivirals (e.g., acyclovir), dexamethasone, immunomodulators (e.g., tacrolimus, leflunomide, cyclophosphamide (Cytoxan®, Neosar®, Endoxan®), and cyclosporine (Neoral®, Sandimmune®, Gengraf®), chlorambucil, azathioprine, methotrexate, and mycophenolate mofetil), biologics (e.g., infliximab (Remicade®), adalimumab (Humira®), etanercept (Enbrel®), golimumab (Simponi®), certolizumab (Cimzia®), rituximab (Rituxan®), abatacept (Orencia®), basiliximab (Simulect®), anakinra (Kineret®), canakinumab (Ilaris®), gevokixumab (XOMA052), tocilizumab (Actemra®), alemtuzumab (Campath®), efalizumab (Raptiva®), LFG316, sirolimus (Santen®), abatacept, sarilumab (Kevzara®), and daclizumab (Zenapax®)), cytotoxic drugs, surgical implant (e.g., fluocinolone insert), and vitrectomy.

In some embodiments, the one or more additional therapeutic agents is selected from an agent/regimen for treating mucositis. Non-limiting examples include AG013, SGX942 (disquietude), amifostine (Ethyol®), cryotherapy, cepacol lonzenges, capsaicin lozenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, Chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble β-1,3/1,6 glucan, P276, LP-0004-09, CR-3294, ALD-518, IZN-6N4, quercetin, granules comprising Vaccinium myrtillus extract, Macleaya cordata alkaloids and Echinacea angustifolia extract (e.g., SAMITAL®), and gastrointestinal cocktail (an acid reducer such aluminum hydroxide and magnesium hydroxide (e.g., Maalox), an antifungal (e.g., nystatin), and an analgesic (e.g., hurricane liquid)). For example, non-limiting examples of treatments for oral mucositis include AG013, amifostine (Ethyol®), cryotherapy, cepacol lonzenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, Chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble β-1,3/1,6 glucan, P276, LP-0004-09, CR-3294, ALD-518, IZN-6N4, quercetin, and gastrointestinal cocktail (an acid reducer such aluminum hydroxide and magnesium hydroxide (e.g., Maalox), an antifungal (e.g., nystatin), and an analgesic (e.g., hurricane liquid)). As another example, non-limiting examples of treatments for esophageal mucositis include xylocaine (e.g., gel viscous Xylocaine 2%). As another example, treatments for intestinal mucositis, treatments to modify intestinal mucositis, and treatments for intestinal mucositis signs and symptoms include gastrointestinal cocktail (an acid reducer such aluminum hydroxide and magnesium hydroxide (e.g., Maalox), an antifungal (e.g., nystatin), and an analgesic (e.g., hurricane liquid)).

In certain embodiments, the one or more additional therapeutic agents is a chemotherapeutic immunomodulator, e.g., an immune checkpoint inhibitor, which can be as defined anywhere herein. In other embodiments, the additional therapeutic agent or regimen is one or more anti-inflammatory agents or immunomodulator acting locally in the GI tract. In other embodiments, the additional therapeutic agent or regimen is 5-ASA (and associated delivery systems), anti-SMAD7 antisense, orally formulated anti-TNFs, anti-integrins, sulfasalazine, balsalazide, steroids, azathioprine, and methotrexate. In further embodiments, the additional therapeutic agent or regimen is radiation or surgery.

In certain embodiments, the one or more additional therapeutic agents is platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

In certain embodiments, the one or more additional therapeutic agents can be selected from those delineated above (see U.S. Pat. No. 7,927,613, which is incorporated herein by reference in its entirety).

In certain embodiments, the one or more additional therapeutic agents can be selected from the compounds that are disclosed generically, sub generically and specifically in any one or more of WO 2004/006906; WO 2006/120178; U.S. 2009/0062396; WO 2012/143377; WO 2012/068274; U.S. Pat. Nos. 7,132,546; 7,989,498; and 8,263,857; each of which is incorporated herein by reference in its entirety.

In certain embodiments, the one or more additional therapeutic agent can be an anthelminthic agent selected from nitazoxanide, closantel, pyrvinium pamoate, and salinomycin. See, e.g., Senkowski, W., et al., Mol. Cancer Ther. 2015, 14, 1504.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### EXAMPLES

### Example 1: Preparation of Enema Formulation Components

The liquid carrier shown in Table E1 below were prepared according to the following procedure. Propyl 4-hydroxybenzoate and methyl 4-hydroxybenzoate were dissolved in hot water. The solution was allowed to cool to room temperature, and additional water was added to compensate water loss due to evaporation that occurred in the prior step. The sodium salts were added and dissolved under stirring for 10 minutes (pH: 6.5-7.5). Methylcellulose and povidone were dispersed using a turbomixer (9000 rpm, 30′). The preparation was allowed to stand for several hours to let foam decant. Typically, the preparation of the liquid carrier was not stored and used immediately. However, when stored, the liquid carriers were stored in 500 mL polyethylene bottles. The liquid carrier exhibited the properties shown in Table E1.

TABLE E1

| Components | Quantity (%) |
|---|---|
| Methyl cellulose (Methocel A15C | 1.40 |
| premium) | |
| Povidone (Kollidon K30) | 1.00 |
| Propyl parahydroxybenzoate | 0.02 |
| Methyl parahydroxybenzoate | 0.20 |
| Disodium phosphate dodecahydrate | 0.15 |
| Sodium dihydrogen phosphate dihydrate | 0.05 |
| Water purified | Up to 100 |
| Technological characterization (as IPC) | |
| Appearance | Clear to opalescent |
| | colloidal dispersion |
| Dynamic viscosity * | 41 mPas s |
| pH | 7.023 |
| Density | 1.0075 g/mL |

The wet granulation preparations shown in Table E2 were prepared according to the following procedure. The internal phase ingredients are combined and mixed in a high-shear granulator. A granulating solution was prepared from water and the indicated agents. This solution is added to the mixture of the inner phase resulting in the formation of granules. Once the granulation was formed and dried, the external phase ingredients were added to the dry granulation. The resultant wet granulation preparations can be suspended in the above-described liquid carriers using conventional procedures.

TABLE E2

| Niclosamide Strength | | 450 mg | | | | | 450 mg |
|---|---|---|---|---|---|---|---|
| | | Component (%) | | | | | |
| Inner phase | Niclosamide | 100 | 98.5 | 77 | 66 | 50 | 61.64 |
| | Colloidal silicon | — | 1.0 | | | | — |
| | dioxide (Aerosil 200) | | | | | | |
| | Magnesium stearate | — | 0.5 | | | | |
| | Cellulose | — | — | 23 | 34 | 50 | — |
| | microcrystalline | | | | | | |
| | (Avicel PH101) | | | | | | |
| | Crospovidone | — | — | — | — | — | 1.92 |
| | (Kollidon CL) | | | | | | |
| | Lactose monohydrate | — | — | — | — | — | 30.82 |
| | (Pharmatose 200M) | | | | | | |
| Granulating | Povidone | — | — | — | — | — | 2.74 |
| solution | (Kollidon K30) | | | | | | |
| | Sodium lauryl sulfate | — | — | — | — | — | 0.68 |
| | Purified water | — | — | — | — | — | * |
| External | Talc | — | — | — | — | — | 1.92 |
| phase | Magnesium stearate | — | — | — | — | — | 0.27 |

| | | | | | | |
|---|---|---|---|---|---|---|
| Theoretical weight (mg) | 450 | 456.9 | 593.4 | 692.3 | 913.8 | 730.0 |

*quantity used: 123 mg/units, removed during the process
Process Parameter

| 1) Calibration step raw materials | Manual calibration | |
|---|---|---|
| 1.1) Calibration sieve | Size 1.0 mm | |
| 2) Mixing step | Turbula, glass container | — |
| 2.1) Mixing time-rotation speed | 5′-34 rmp | — |
| 3) Granulation step | — | Manual |
| | | granulation |
| 3.1) wet granulate sieve | | 1.0 mm |

Technological Characterization

| | Granulate | |
|---|---|---|
| Loss on drying (105° C. for 10′) | — | 1.4% |

| | | Final mix | |
|---|---|---|---|
| Flowability* | 10.0 | It did not pass | It did not pass |
| Flow throw an orifice of | 15.0 | It did not pass | 6.1 g/sec |
| ∅(mm): | 25.0 | It did not pass | 17.8 g/sec |

| | | |
|---|---|---|
| Suspendability | Not homogeneous suspendability and | Rapid and |
| | very poor mixture wettability | Homogeneous |
| | | pH 6.9 |

* 100 g of granulate have to pass through an orifice of increasing size 10 or 15 or 25 (etc.) mm
diameter and the size of the orifice is increased if the powder is not passing through. When it
passes the time is taken so that the smaller the diameter of the orifice and higher the
amount/second the better it is for the flow properties of the granulate.
Analytical test

| | | |
|---|---|---|
| Niclosamide assay (%) | — | 58.84% |

### Example 2: Phase 1/2a Clinical Study of Niclosamide Enema in Subjects with Mild-to-Moderate UP or UPS with Inadequate Response to 5-ASA

This example describes a Phase 1/2a multi-center, open-label, single-arm, sequential cohort study aimed at evaluating safety, efficacy, and the pharmacokinetics of Niclosamide Enema in subjects with mild-to-moderate ulcerative proctitis (UP) and ulcerative proctosigmoiditis (UPS).

As used in this example, Niclosamide Enema refers to the formulation described in Example 1.

### Study Design

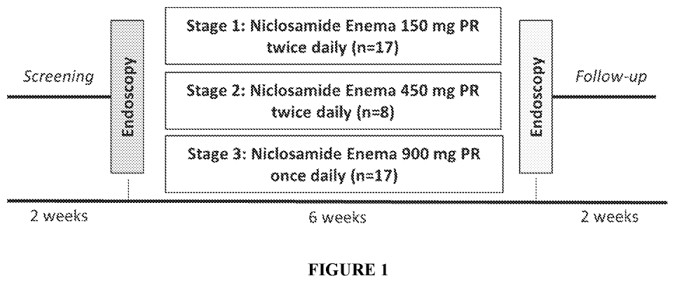

FIG. 1 schematically describes the design of the clinical study.

### Inclusion Criteria

1) Diagnosis of ulcerative proctitis (UP) or proctosigmoiditis (UPS) at least 3 months prior to treatment;
2) Mild-to-moderate disease activity, defined as composite score (modified Mayo score) ≥4 to <8; and
3) Inadequate response to first-line therapy, 5-ASAs.

### Dosing

Stage 1: 17 subjects were dosed with 150 mg of Niclosamide Enema (i.e., 150 mg niclosamide) per the rectum (PR) twice daily for 6 weeks.
Stage 2: 8 subjects are dosed with 450 mg of Niclosamide Enema PR twice daily for 6 weeks.
Stage 3: 17 subjects are dosed with 900 mg of Niclosamide Enema PR once daily for 6 weeks.

### Assessment Criteria

Safety
Pharmacokinetics
Symptom scores (stool frequency, rectal bleeding)
Endoscopy, with intestinal mucosal biopsies for histologic assessment, cytokine expression

### Results

Safety Tolerability: All 17 subjects in Stage 1 completed the 6-week treatment period. Treatment-emergent adverse event (TEAE) was reported in 35% of the subjects (6/17 subjects). TEAE was mild in 5 of the 6 subjects. In all 17 subjects, no serious or drug-related TEAEs were reported. No discontinuations resulting from TEAEs were reported.

Efficacy: Clinical remission (MMS≤2 with no individual subscore >1) was reported for 59% of the subjects (10/17 subjects). Clinical response (decrease in MMS≥2; decrease in MMS≥25%; decrease in RBS≥1; and RBS=0 or 1) was reported for 65% of the subjects (11/17 subjects). 58% of the subjects (7/12 subjects) exhibited endoscopic remission (modified endoscopic subscore of 0 or 1, infra, Table E3). Further, 67% of the subjects (4/6 subjects) with baseline fecal calprotectin (FC)>250 μg/g showed FC<250 μg/g at week 6. Additional details are provided in Table E3.

TABLE E3
Efficacy of Niclosamide Enema Treatment in Stage 1 Subjects

| Efficacy | Definition | Result |
|---|---|---|
| Clinical | *MMS < 2 with no individual | 59% (10/17) |
| remission | subscore > 1 | |
| Clinical | Decrease in MMS ≥ 2 and decrease in | 65% (11/17) |
| response | MMS ≥ 25% and decrease in **RBS ≥ 1 | |
| | and RBS 0 or 1 | |
| Endoscopic | Modified endoscopic subscore of 0 or 1; | 58% (7/12) |
| remission | score of 1 excludes friability. | |
| | Excludes subjects meeting criteria for | |
| | remission at baseline. | |

*MMS = modified Mayo score
**RBS = Rectal Bleeding Score

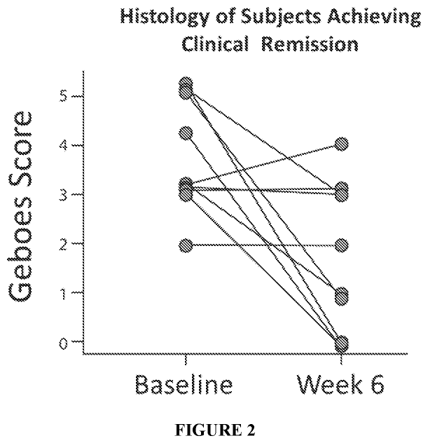

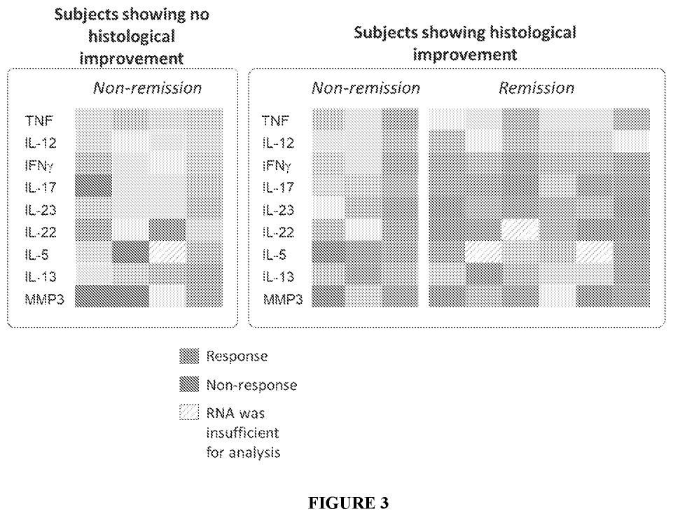

Post-Hoc Analysis of Histologic Improvements and Endoscopic Mucosal Healing Histologic remission has been consistently associated with lower rates of relapse, corticosteroid dependence, hospitalization, and colorectal cancer than endoscopic remission alone in observational studies (see Am. J. Gastroenterol. 2019; 144:733 which is incorporated herein by reference in its entirety). Following Stage 1 treatment, members of the 17 subjects who did not meet the criteria for histologic improvement or endoscopic healing at baseline were analyzed for histologic improvement (by Geboes Score) and endoscopic mucosal healing. The basis for the post-hoc analysis and findings are summarized in Table E4. FIG. 2 shows the Geboes Score of subjects achieving clinical remission at week 6 relative to the baseline values

TABLE E4
Post-Hoc Analyses

| Assessment | Basis | Finding |
|---|---|---|
| Histologic | Defined as “histologic healing” in Sands | 50% |
| improvement | NEJM 2019 and “histologic improvement” | (7/14) |
| | in Li J. Crohn's Colitis 2019; Excludes | |
| | subjects meeting criteria for histologic | |
| | improvement at baseline | |
| Endoscopic | Defined as in Sands NEJM 2019; Excludes | 40% |
| mucosal | subjects meeting criteria for endoscopic | (6/15) |
| healing | healing at baseline | |

In addition, 13 of the 17 Stage 1 subjects were analyzed for the expression of colonic mucosal cytokines TNF, IL-12, IFNγ, IL-17, IL-23, IL-22, IL-5, IL-13, and MMP3. FIG. 3 provides a heat-map of the expression of cytokines in subjects treated with Niclosamide Enema compared to the expression level of these subjects at the baseline. As can be seen, decreased levels of inflammatory cytokines were observed in the colonic mucosa of each subject.

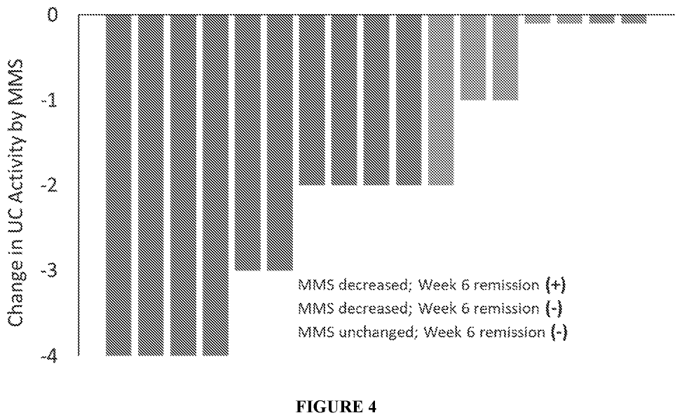

Overall, changes in MMS, histology, and/or cytokine expression were observed in 16 of 17 the subjects for Stage 1. As illustrated by FIG. 4, the 10 subjects in clinical remission exhibited decreases in MMS; and decreases in MMS were also observed for 3 of the 7 subjects that did not show clinical remission. While the remaining 4 subjects did not show observable improvements in MMS following treatment for 6 weeks, 3 of these 4 subjects exhibited decreases in histologic score and decreases in mucosal cytokine expression (see Table E5).

TABLE E5
Histologic Score and Mucosal Cytokine Expression in Stage
1 Subjects that did not Exhibit Improvements in MMS

| Subject | ΔHistology | ΔIFNγ | ΔIL-17 | ΔIL-23 | ΔTNFα |
|---|---|---|---|---|---|
| 58 yo (Woman UPS) | 5.3 → 3.1 | −72% | −66% | −86% | −72% |
| 34 yo (Man UPS) | 5.2 → 4.3 | −35% | −26% | 9% | −37% |
| 40 yo (Woman UP) | 5.2 → 5.1 | −8% | −15% | 20% | −38% |
| 26 yo (Woman UPS) | 3.1 → 5.3 | −5% | −12% | 21% | 34% |

Without wishing to be bound by theory, it is believed that changes in proinflammatory cytokines and histology precede mucosal healing, suggesting that higher rates of remission may be achieved with a longer duration of treatment.

A number of embodiments of the invention have been described. Nevertheless, it will be understood that various modifications may be made without departing from the spirit and scope of the invention. Accordingly, other embodiments are within the scope of the following claims.

1. A method for treating colitis in a subject in need thereof, the method comprising administering by enema an effective amount of a formulation comprising a first component and a second component, wherein:
(i) the first component comprises a solid pharmaceutical composition, which comprises:
an inner phase which is a wet granulated solid preparation comprising niclosamide, or a pharmaceutically acceptable salt thereof, one or more disintegrants; one or more diluents; and one or more binders;
an external phase comprising one or more glidants and/or one or more lubricants; and
(ii) the second component comprises one or more liquids and optionally one or more other pharmaceutically acceptable excipients together forming a liquid carrier,
wherein the subject has a modified Mayo score (MMS) equal to from 4 points or greater than 4 points to 8 points or less than 8 points prior to said administering.

2. A method for treating colitis in a subject in need thereof, the method comprising administering by enema an effective amount of a formulation comprising a first component and a second component, wherein:
(i) the first component comprises a solid pharmaceutical composition, which comprises:
an inner phase which is a wet granulated solid preparation comprising niclosamide, or a pharmaceutically acceptable salt thereof, one or more disintegrants; one or more diluents; and one or more binders;
an external phase comprising one or more glidants and/or one or more lubricants; and
(ii) the second component comprises one or more liquids and optionally one or more other pharmaceutically acceptable excipients together forming a liquid carrier,
wherein the subject has at least one of:
(I) a modified Mayo score (MMS) following said administering that is at least 2 points lower than a modified Mayo score (MMS) in the subject prior to said administering;
(II) a modified Mayo score (MMS) following said administering that is at least 25% lower than a modified Mayo score (MMS) in the subject prior to said administering;
(III) a Rectal Bleeding Score (RBS) following said administering that is at least 1 point lower than a Rectal Bleeding Score (RBS) in the subject prior to said administering; or
(IV) a Rectal Bleeding Score (RBS) following said administering that is 0 or 1.

3. (canceled)

4. (canceled)

5. A method for treating colitis in a subject in need thereof, the method comprising administering by enema an effective amount of a formulation prepared by mixing together a second component, wherein:
(i) the first component comprises a solid pharmaceutical composition, which comprises:
an inner phase which is a wet granulated solid preparation comprising niclosamide, or a pharmaceutically acceptable salt thereof; one or more disintegrants; one or more diluents; and one or more binders;
an external phase comprising one or more glidants and/or one or more lubricants; and
(ii) the second component comprises one or more liquids and optionally one or more other pharmaceutically acceptable excipients together forming a liquid carrier,
wherein following said administering the subject shows a decrease in the level of an inflammatory marker relative to the level of the inflammatory marker prior to said administering.

6. (canceled)

7. (canceled)

8. (canceled)

9. (canceled)

10. (canceled)

11. (canceled)

12. (canceled)

13. (canceled)

14. (canceled)

15. The method of claim 1,
wherein following said administering the subject shows a decrease in the level of an inflammatory marker relative to the level of the inflammatory marker prior to said administering.

16. (canceled)

17. (canceled)

18. (canceled)

19. (canceled)

20. (canceled)

21. (canceled)

22. (canceled)

23. The method of claim 5, wherein the level of the inflammatory marker is the level of the marker in colonic mucosa.

24. The method of claim 5, wherein the level of the inflammatory marker is the level of the marker in plasma.

25. The method of claim 5, wherein the inflammatory marker is TNFα.

26. The method of claim 5, wherein the inflammatory marker is IFNγ.

27. The method of claim 5, wherein the inflammatory marker is selected from the group consisting of IL-12, IL-17, IL-22, IL-23, IL-5, and IL-13.

28. The method of claim 5, wherein the inflammatory marker is IL-12.

29. The method of claim 5, wherein the inflammatory marker is IL-17.

30. The method of claim 5, wherein the inflammatory marker is IL-23.

31. The method of claim 5, wherein the inflammatory marker is IL-22.

32. The method of claim 5, wherein the inflammatory marker is IL-5.

33. The method of claim 5, wherein the inflammatory marker is IL-13.

34. The method of claim 5, wherein the inflammatory marker is MMP3.

35. The method of claim 5,
wherein the decrease in the level of the inflammatory marker following said administering relative to the level of the inflammatory marker prior to said administering is of at least 5%, such as at least 10%, such as at least 15%, such as at least 20%, such as at least 30%, such as at least 40%, such as at least 50%, such as at least 60%, such as at least 70%, such as at least 80%.

36. (canceled)

37. (canceled)

38. (canceled)

39. (canceled)

40. (canceled)

41. (canceled)

42. The method of claim 1, wherein upon administration, the local concentration of niclosamide in the GI tract is higher than the concentration of niclosamide in the plasma compartment.

43. The method of claim 42, wherein upon administration, the local concentration of niclosamide in the GI tract is about 10 times higher than the concentration of niclosamide in the plasma compartment.

44. The method of claim 42, wherein niclosamide in the plasma compartment is subject to first pass metabolism.

45-78. (canceled)
